(19) United States
(12) Patent Application Publication
(10) Pub. No.: US 2021/0381058 A1
(43) Pub. Date: Dec. 9, 2021

(54) EVIDENCE BASED SELECTION OF PATIENTS FOR CLINICAL TRIALS USING HISTOPATHOLOGY

(71) Applicant: Nantomics, LLC, Culver City, CA (US)

(72) Inventors: Christopher W. Szeto, Culver City, CA (US); Stephen Charles Benz, Culver City, CA (US)

(21) Appl. No.: 17/281,944

(22) PCT Filed: Sep. 23, 2019
(86) PCT No.: PCT/US2019/052456

§ 371 Date: Mar. 31, 2021

Related U.S. Application Data
(60) Provisional application No. 62/739,551, filed on Oct. 1, 2018.

(51) Int. Cl. C12Q 1/6886; G16B 25/10; G16B 20/00; G16H 50/70; G16H 10/20; G16H 50/20

(52) U.S. Cl. CPC C12Q 1/6886; G16B 25/10; G16B 20/00; C12Q 2600/158; G16H 10/20; G16H 50/20; C12Q 2600/112; G16H 50/70

(57) ABSTRACT

An immune gene expression signature and immune cell distribution in the tumor, in combination, can be used to infer an immune phenotype of the tumor, which further can be used to characterize the tumor, selecting an optimal immune therapy to the tumor, and predicting the treatment outcome of an immune therapy.

This application claims priority to our copending U.S. provisional application Ser. No. 62/739,551, which was filed Oct. 1, 2018, and which is incorporated by reference herein.

### FIELD OF THE INVENTION

The field of the invention is combinatorial genetic analysis and histological analysis of tumor tissue, especially as it relates to immune cells signatures.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Studies of the tumor microenvironment have surfaced promising avenues of exploration to better understand the clinical relevance of various immune cells in the tumor microenvironment and their interactions with the tumor cells. Yet, immune heterogeneity within the tumor microenvironment has added multiple layers of complexity to the understanding of chemosensitivity and survival across various cancer types. For example, within the tumor microenvironment, immunogenicity is a favorable clinical feature in part driven by the antitumor activity of CD8+ T cells. However, tumors often inhibit this antitumor activity by exploiting the suppressive function of Regulatory T cells (Tregs), thus suppressing an adaptive immune response.

In addition, there are numerous mechanisms other than Tregs and CD8+ T involved in the immunogenicity of tumor cells, and an accurate prediction of immunogenicity of a tumor has remained elusive. Indeed, it has been reported that the immune infiltrate composition changes at each tumor stage and that particular immune cells have a major impact on survival. For example, densities of T follicular helper (Tfh) cells and innate cells increases, and most T cell densities decrease where tumor progression is observed. Moreover, the number of B cells, which are key players in the core immune network and are associated with prolonged survival, increase at a late stage and often show a dual effect on recurrence and tumor progression (see e.g., Immunity 2013 Oct. 17; 39(4):782-95).

On that background, some tried to identify and utilize specific gene signatures and histopathologic data of the tumor tissue as clinical parameters for a tumor tissue. For example, U.S. Pat. No. 9,404,926 to Mule discloses that expression levels of certain chemokines, cytotoxic genes, and/or dendritic cell genes can be used to determine a molecular signature of the colorectal cancer tissue. In addition, Mule further discloses that histopathologic data of immune cells in the tumor tissue as a corroborative data to associate the tumor tissue structure and the chemokine expressions. In another example, Galon et al., (Science, Vol. 313, 1960-1964, 29 Sep. 2006) discloses correlation analyses of 18 immunogenes in colorectal cancer tissue to classify the tissue into three immune status categories, and found that tissues showing Th1 adaptive immunity is associated with better prognosis of the disease. Galon further discloses that the number and location of T cells in the tumor tissue may be a critical indicator to the prognosis of disease to so predict the survival time based on such information. However, these attempts are limited to a limited number of specific genes that are known to be related general immunogenicity of tumor cells and often are not specifically related to the immune cell types. In addition, all such arts merely use the histopathological data obtained from the tumor tissue as a mere confirmatory tool or as an independent tool to characterize immune status of the tumor tissue, and thus fail to use these analyses in conjunction with the transcriptomics data.

Therefore, despite numerous findings in isolation, complex interactions between tumors and immune cells in the microenvironment remain to be elucidated. Consequently, there is still a need for improved systems and methods to better characterize immunogenicity of a tumor.

### SUMMARY OF THE INVENTION

The inventive subject matter is directed to various methods of genetic analysis of tumor tissue, and advantageously allows for examination and identification of immune cells in the tumor microenvironment as well as their activities to so further enable infer the immune phenotype of the tumor tissue. Thus, one aspect of the inventive subject matter includes a method of characterizing a tumor. In this method, expression levels for a plurality of distinct genes in the tumor is quantified or obtained. The distinct genes are associated with respective distinct types of immune cells. Also, distribution of at least one type of the immune cells in the tumor is determined. Then, an immune phenotype of the tumor can be inferred based on the expression levels for the plurality of distinct genes and the distribution of the at least one type of the immune cells. Most typically the distinct immune cells in the tumor are selected from pDC, aDC, TfH, NK cells, neutrophils, Treg, iDC, macrophages, T helper cells, CD8 T cells, CD4 T cells.

Typically, the expression level is measured via qPCR or RNAseq. In some embodiments, over-expression or under-expression for each of the distinct genes can be determined relative to respective reference ranges, wherein the reference ranges are specific for a specific tumor type. In such embodiment, the over-expression or under-expression can be determined when the quantified expression level exceeds +/−2 SD of the reference range. In addition, it is contemplate that the reference ranges are specific for a specific tumor type as classified in ICD10. With respect to the distribution of at least one type of the immune cells, the distribution can be determined from immunolabeling or in situ hybridization of the tumor.

Optionally, the method can further continue with a step of obtaining at least two of genomics, transcriptomics, and proteomics data from tumor cells in the tumor and inferring a pathway characteristic of the tumor cells. Then, based on the pathway characteristic, a molecular signature of the tumor cells can be assigned. In such embodiment, the immune phenotype of the tumor can be further inferred based on the molecular signature in combination with expression levels for the plurality of distinct genes and the distribution of the at least one type of the immune cells. The pathway characteristic may comprise a constitutively activated pathway, a functionally impaired pathway, and a dysregulated pathway and/or an immune-inhibitory pathway and immune-resistant pathway. Preferably, the pathway characteristic is inferred using PARADIGM.

It is contemplate that the immune phenotype inferred by the inventive subject matter include immune desert, immune periphery, and immune infiltration. Once the immune phenotype is identified, then the method may further comprise a step of generating or updating a treatment regimen to the patient. In one embodiment, treatment regimen includes a checkpoint inhibitor when the immune phenotype is determined to be immune desert. In such embodiments, the checkpoint inhibitor can be a PD-L1/PD-1 inhibitor, a TIM3 inhibitor or an IDO inhibitor. In other embodiments, the treatment regimen includes a chemokine to recruit T cells when the immune phenotype is determined to be immune periphery, and the chemokine can include VEGF or CXCR4.

In another aspect of inventive subject matter, the inventors contemplate a method of predicting treatment outcome for immune therapy of a tumor. In this method, expression levels for a plurality of distinct genes in the tumor is quantified or obtained. The distinct genes are associated with respective distinct types of immune cells. Also, distribution of at least one type of the immune cells in the tumor is determined. Then, an immune phenotype of the tumor can be inferred based on the expression levels for the plurality of distinct genes and the distribution of the at least one type of the immune cells. Based on the inferred immune phenotype, a likelihood of success of the immune therapy can be predicted.\ Most typically the distinct immune cells in the tumor are selected from pDC (plasmacytoid dendritic cell), aDC (activated dendritic cell), TfH (T follicular helper cell), NK cells, neutrophils, Treg, iDC (immature dendritic cells), macrophages, T helper cells, CD8+ T cells, CD4+ T cells.

Typically, the expression level is measured via qPCR or RNAseq. In some embodiments, over-expression or under-expression for each of the distinct genes can be determined relative to respective reference ranges, wherein the reference ranges are specific for a specific tumor type. In such embodiment, the over-expression or under-expression can be determined when the quantified expression level exceeds +/−2 SD of the reference range. In addition, it is contemplate that the reference ranges are specific for a specific tumor type as classified in ICD10. With respect to the distribution of at least one type of the immune cells, the distribution can be determined from immunolabeling or in situ hybridization of the tumor.

Optionally, the method can further continue with a step of obtaining at least two of genomics, transcriptomics, and proteomics data from tumor cells in the tumor and inferring a pathway characteristic of the tumor cells. Then, based on the pathway characteristic, a molecular signature of the tumor cells can be assigned. In such embodiment, the immune phenotype of the tumor can be further inferred based on the molecular signature in combination with expression levels for the plurality of distinct genes and the distribution of the at least one type of the immune cells. The pathway characteristic may comprise a constitutively activated pathway, a functionally impaired pathway, and a dysregulated pathway and/or an immune-inhibitory pathway and immune-resistant pathway. Preferably, the pathway characteristic is inferred using PARADIGM.

It is contemplate that the immune phenotype inferred by the inventive subject matter include immune desert, immune periphery, and immune infiltration. It is contemplated that in some embodiments, the immune therapy comprises treatment with a checkpoint inhibitor and the likelihood of success of the immune therapy is high if the immune phenotype is determined to be immune desert. In other embodiments, the immune therapy comprises treatment with at least one of a vaccine composition and an immune stimulatory cytokine, and the likelihood of success of the immune therapy is high if the immune phenotype is determined to be immune periphery.

In still another aspect of the inventive subject matter, the inventors contemplate a method of treating a patient having a tumor. In this method, expression levels for a plurality of distinct genes in the tumor is quantified or obtained. The distinct genes are associated with respective distinct types of immune cells. Also, distribution of at least one type of the immune cells in the tumor is determined. Then, an immune phenotype of the tumor can be inferred based on the expression levels for the plurality of distinct genes and the distribution of the at least one type of the immune cells. The patient can be treated with an immune therapy selected based on the immune phenotype. Most typically the distinct immune cells in the tumor are selected from pDC, aDC, TfH, NK cells, neutrophils, Treg, iDC, macrophages, T helper cells, CD8 T cells, CD4 T cells.

Typically, the expression level is measured via qPCR or RNAseq. In some embodiments, over-expression or under-expression for each of the distinct genes can be determined relative to respective reference ranges, wherein the reference ranges are specific for a specific tumor type. In such embodiment, the over-expression or under-expression can be determined when the quantified expression level exceeds +/−2 SD of the reference range. In addition, it is contemplate that the reference ranges are specific for a specific tumor type as classified in ICD10. With respect to the distribution of at least one type of the immune cells, the distribution can be determined from immunolabeling or in situ hybridization of the tumor.

Optionally, the method can further continue with a step of obtaining at least two of genomics, transcriptomics, and proteomics data from tumor cells in the tumor and inferring a pathway characteristic of the tumor cells. Then, based on the pathway characteristic, a molecular signature of the tumor cells can be assigned. In such embodiment, the immune phenotype of the tumor can be further inferred based on the molecular signature in combination with expression levels for the plurality of distinct genes and the distribution of the at least one type of the immune cells. The pathway characteristic may comprise a constitutively activated pathway, a functionally impaired pathway, and a dysregulated pathway and/or an immune-inhibitory pathway and immune-resistant pathway. Preferably, the pathway characteristic is inferred using PARADIGM.

It is contemplate that the immune phenotype inferred by the inventive subject matter include immune desert, immune periphery, and immune infiltration. It is contemplated that in some embodiments, immune therapy includes a checkpoint inhibitor when the immune phenotype is determined to be immune desert, and the checkpoint inhibitor may include a PD-L1/PD-1 inhibitor, a TIM3 inhibitor or an IDO inhibitor. In other embodiments, the immune therapy includes a chemokine to recruit T cells when the immune phenotype is determined to be immune periphery, and the chemokine may include VEGF or CXCR4.

Still another inventive subject matter include use of transcriptomics data of a plurality of distinct genes and immune cell distribution data of a tumor of a patient to characterize a tumor, to predict treatment outcome for immune therapy of the tumor, or to treat the patient. Most preferably, the use comprises inferring an immune phenotype of the tumor based on the transcriptomics data and immune cell distribution data. Typically, the distinct immune cells in the tumor are selected from pDC, aDC, TfH, NK cells, neutrophils, Treg, iDC, macrophages, T helper cells, CD8 T cells, CD4 T cells, and/or the transcriptomics data comprise expression levels for the plurality of distinct genes obtained using RNA-seq. The immune cell distribution data is obtained from immunolabeling or in situ hybridization of the tumor.

Various objects, features, aspects and advantages of the inventive subject matter will become more apparent from the following detailed description of preferred embodiments, along with the accompanying drawing.

### BRIEF DESCRIPTION OF THE DRAWING

FIG. 1 is an exemplary table of immune cells and genes specific or characteristic of the immune cells.

FIG. 2 depicts RNAseq expression of genes in the immune cell panel of FIG. 1 in 1037 clinical cases.

FIG. 3 exemplarily depicts immune cell category activation by tissue-type.

FIGS. 4A-4H illustrate exemplary immune cell category activation by tissue-type distributions.

FIG. 5 is a table listing statistics for each cancer type.

FIG. 6 is an exemplary report showing sample-specific high/normal/low calls, and z-scores, are given for each cell type.

FIG. 7 shows exemplary checkpoint expression patterns.

FIG. 8 depicts exemplary immune-cell activation in PDL1 categories, allowing for a determination as to whether tissue samples are enriched or suppressed in those cell types.

FIG. 9 depicts exemplary photomicrographs with high specificity between deep-net generated tumor masks and pathologist annotations.

FIG. 10 depicts exemplary graphs related to alignment of purity and stromal estimates with expectations from DNA & RNA.

FIG. 11 depicts exemplary results for RNAseq-based immune deconvolution sorted by sTIL levels.

FIG. 12 depicts exemplary results for RNAseq-based lymphocyte score vs. various deep-net assessments.

FIG. 13 depicts exemplary results for checkpoint expression patterns using different methods of bifurcating patients.

FIG. 14 depicts exemplarily results illustrating which immune-cell types are most associated with sTIL levels.

### DETAILED DESCRIPTION

The inventors contemplate that distribution and number of immune cell types in a tumor microenvironment, individually or in combination, contribute to shape the immune-susceptible or immune-resistant phenotype of the tumor microenvironment. In other words, such elements may influence tumor microenvironment differently or represent different condition of tumor microenvironment depending on what other elements are present. Thus, it is often necessary to conduct a comprehensive screening of multiple elements and combinatorial analysis of such elements.

Viewed from a different perspective, the inventors discovered that more accurate representation of the tumor microenvironment's immune status, or immune phenotype of the tumor microenvironment, can be readily and comprehensively determined or inferred using two or more molecular and/or cellular signatures of the tumor microenvironment and/or the cells present in the tumor microenvironment. The inventors also found that such inferred immune phenotype can be reliably used to generate treatment regimen including immune therapy or predict the outcome of the immune therapy. Consequently, in one especially preferred aspect of the inventive subject matter, the inventors contemplate a method of characterizing a tumor by inferring an immune phenotype of the tumor based on the expression levels for the plurality of distinct genes and the number, type, and/or distribution of at least one type of immune cells in the tumor.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

### Obtaining Omics Data

Any suitable methods and/or procedures to obtain omics data are contemplated. For example, the omics data can be obtained by obtaining tissues from an individual and processing the tissue to obtain DNA, RNA, protein, or any other biological substances from the tissue to further analyze relevant information. In another example, the omics data can be obtained directly from a database that stores omics information of an individual.

Where the omics data is obtained from the tissue of an individual, any suitable methods of obtaining a tumor sample (tumor cells or tumor tissue) or healthy tissue from the patient are contemplated. Most typically, a tumor sample or healthy tissue sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, tissues or cells may be fresh or frozen. In other example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a healthy tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or healthy tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

Where it is desired to obtain the tumor-specific omics data, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV),VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, omics data analysis may reveal missense and nonsense mutations, changes in copy number, loss of heterozygosity, deletions, insertions, inversions, translocations, changes in microsatellites, etc.

Moreover, it should be noted that some data sets are preferably reflective of a tumor and a matched normal sample of the same patient to so obtain patient and tumor specific information. In such embodiments, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

In addition, omics data of cancer and/or normal cells comprises a transcriptome data set that includes sequence information and expression level (including expression profiling, copy number, or splice variant analysis) of RNA(s) (preferably cellular mRNAs) that is obtained from the patient, from the cancer tissue (diseased tissue) and/or matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyAtRNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in US 9824181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

It should be appreciated that one or more desired nucleic acids or genes may be selected for a particular disease (e.g., cancer, etc.), disease stage, specific mutation, or even on the basis of personal mutational profiles or presence of expressed neoepitopes. Alternatively, where discovery or scanning for new mutations or changes in expression of a particular gene is desired, RNAseq is preferred to so cover at least part of a patient transcriptome. Moreover, it should be appreciated that analysis can be performed static or over a time course with repeated sampling to obtain a dynamic picture without the need for biopsy of the tumor or a metastasis.

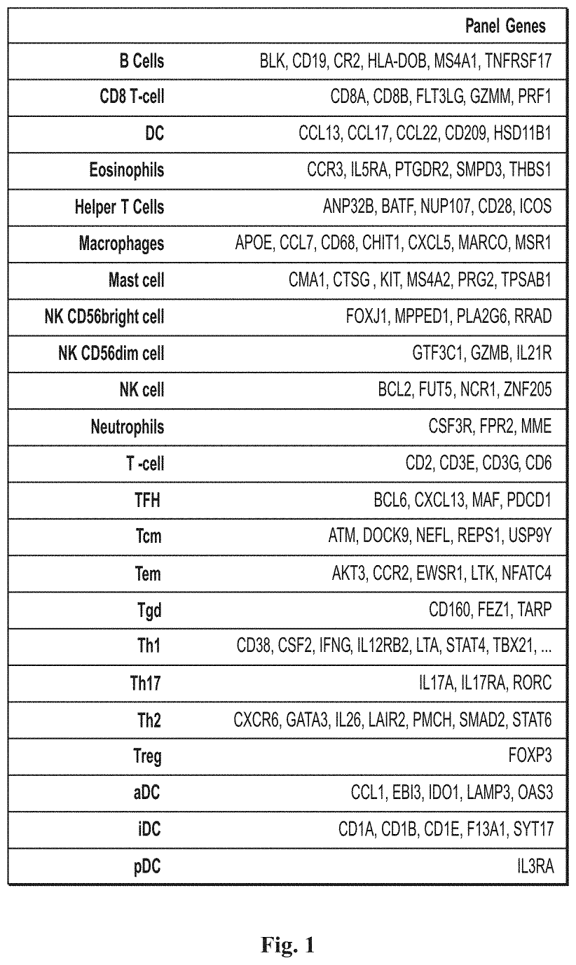

While any suitable set of genes for the transcriptomics data are contemplated, a preferred gene set to analyze the tumor microenvironment's immune status or immune phenotype includes any genes identified as being associated or characteristic for a specific immune cell type. An exemplary set of genes is shown in FIG. 1, which lists cell types (e.g., pDC, aDC, TfH, NK cells, neutrophils, Treg, iDC, macrophages, T helper cells, CD8+ T cells, CD4+ T cells, etc.) by role and function and specific genes associated with these cell types. In other embodiments, the gene set may include genes that may indicate the immune status of the tumor tissue (e.g., immune-suppressed, inflammatory, helper T adaptive immunity-active, etc.). Exemplary gene set in such embodiments include, but not limited to, MMP-7, PTGS2, IL-8, BIRC5, CEACAM1, GZMB, GLNU, IFNG, IRF1, CD3z, CD8a, TBX21, TNFRSF10A, B7H3, CD4, IL10, TGFB1, and VEGF.

Further, omics data of cancer and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

### Transcriptomics Data Analysis and Molecular Signature

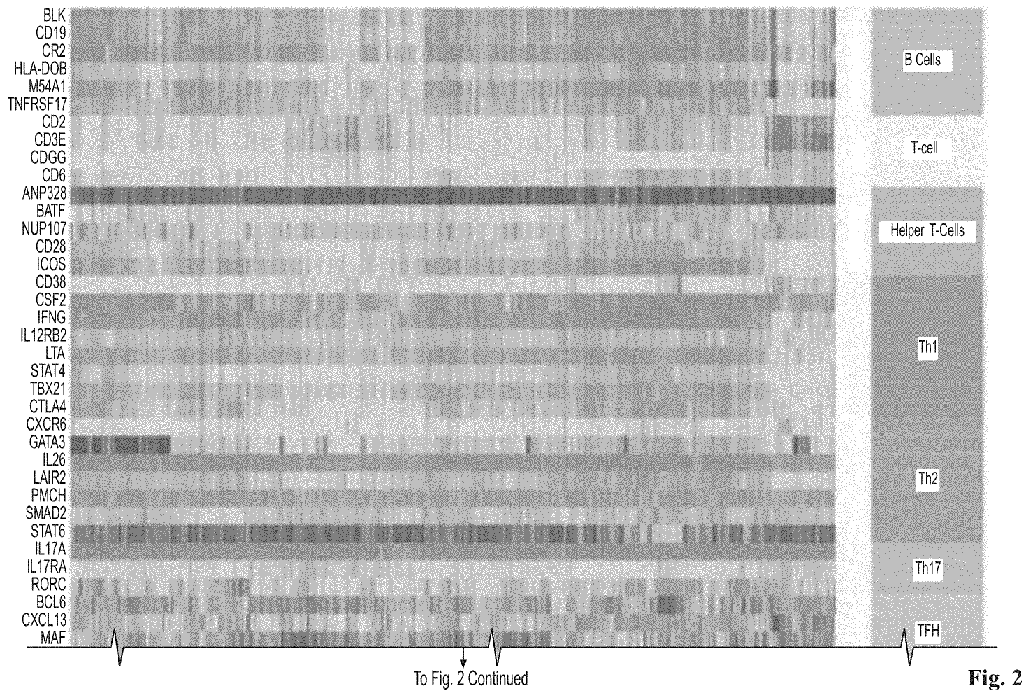

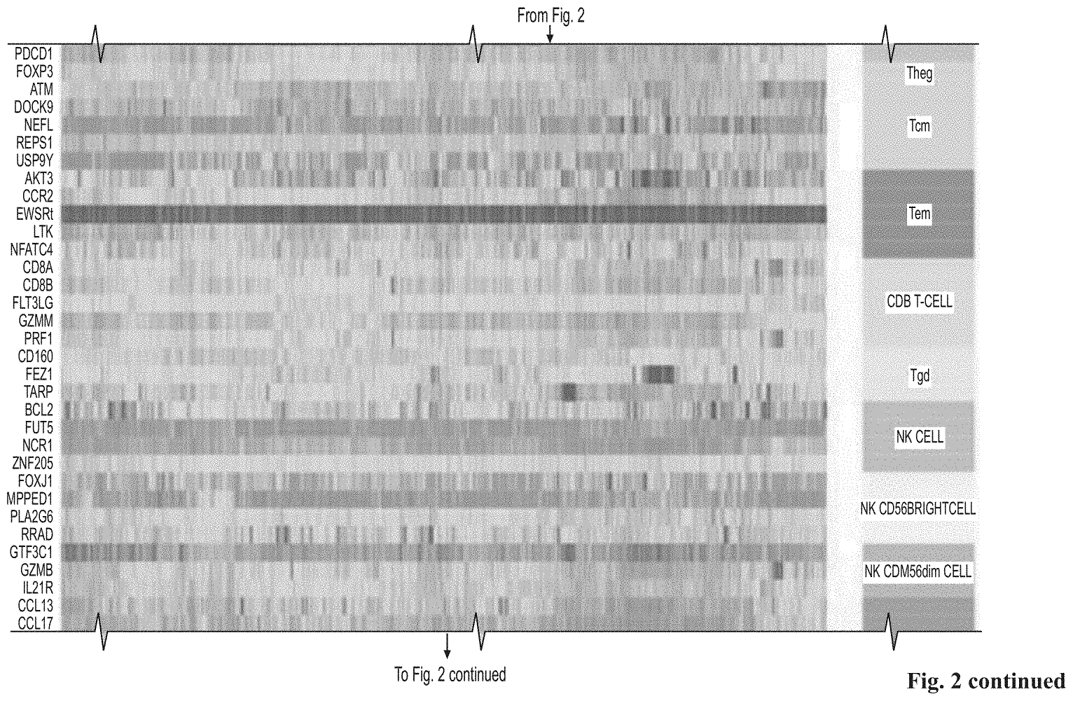

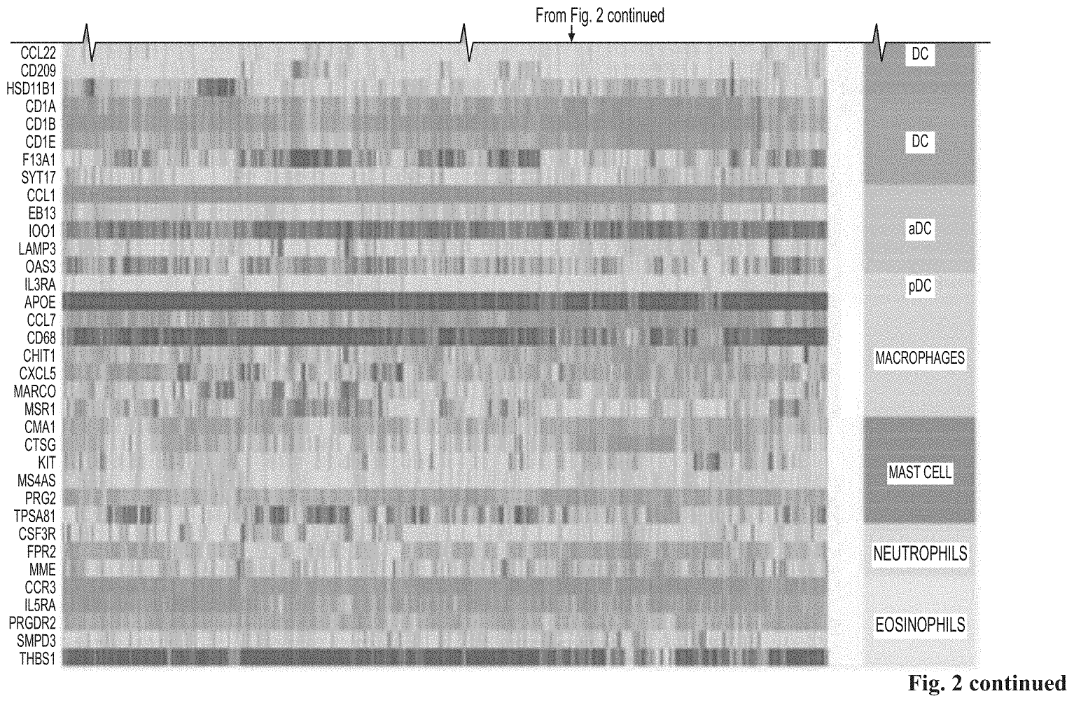

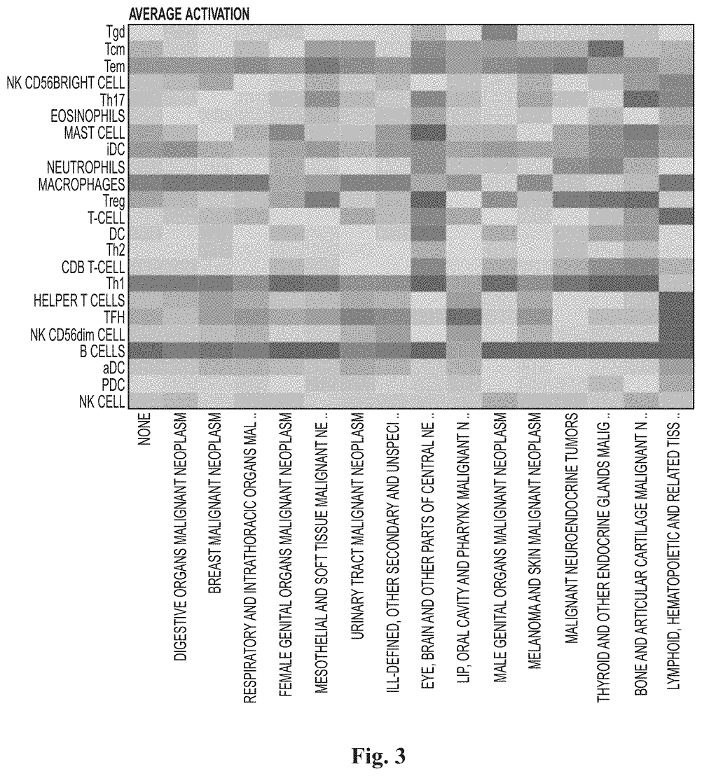

The inventors contemplate that transcriptomics data from the tumor sample, especially the expression levels of selected genes that are associated with the immune cell types can be used to predict or infer the types of immune cell and their activity levels in the tumor tissue. In some embodiments, transcriptomics data obtained from RNA-seq of the selected genes may be clustered into several groups, each of the group corresponds to a type of immune cells or an activity of the immune cell. In other embodiments, transcriptomics data obtained from RNA-seq of the selected genes may be clustered into several groups, in which each of the groups corresponds to specific inferred immune status of the tumor tissue (e.g., immune-suppressed, inflammatory, helper T adaptive immunity-active, etc.). For example, the inventors investigated whether expression levels of these genes would cluster, using RNAseq performed on 1037 tumor samples. FIG. 2 depicts an exemplary result where the rows are ordered by immune cell categories per FIG. 1, and where the columns are ordered by hierarchical clustering using Pearson similarity score. Colors range from blue (log2[TPM+1]=0) to red (log2[TPM+1]˜12.5). When expression of the immune genes for each immune cell type was averaged, and when the average values were correlated with different cancer types, specific signatures became apparent as is exemplarily illustrated in FIG. 3. Here, the heat map shows an average expression for all genes in each immune cell category, split up into reported ICD10 categories. Rows are ordered by hierarchical clustering (using Pearson similarity score), and the columns are ordered from left-to-right by how many samples were annotated for that cancer type. Colors range from blue (avg. log2[TPM+1]˜0.35) to red (avg. log2[TPM+1]˜5.0).

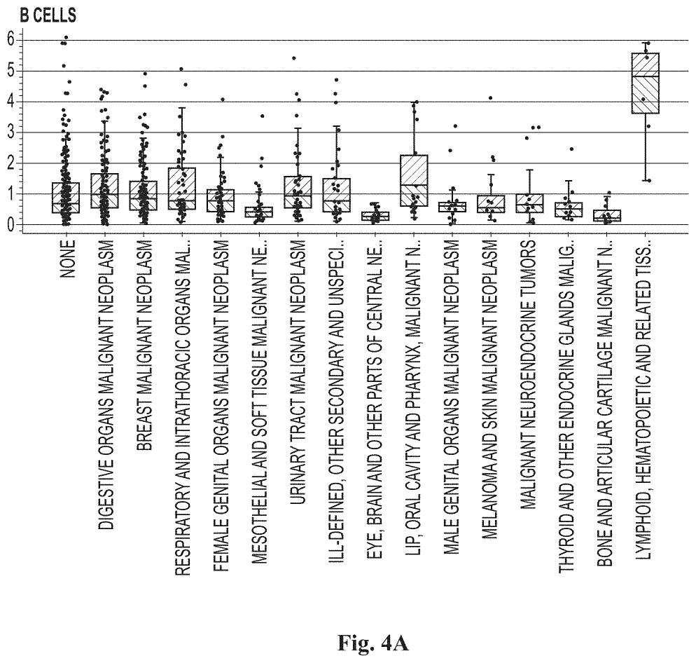

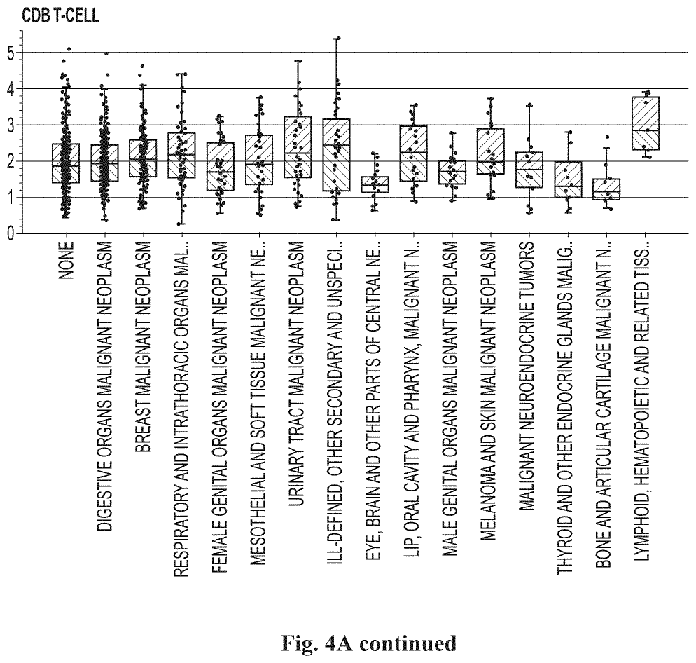

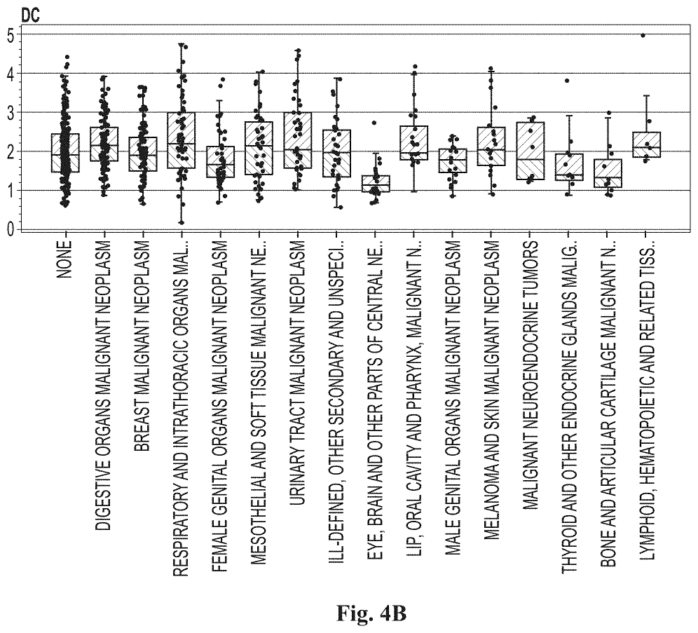

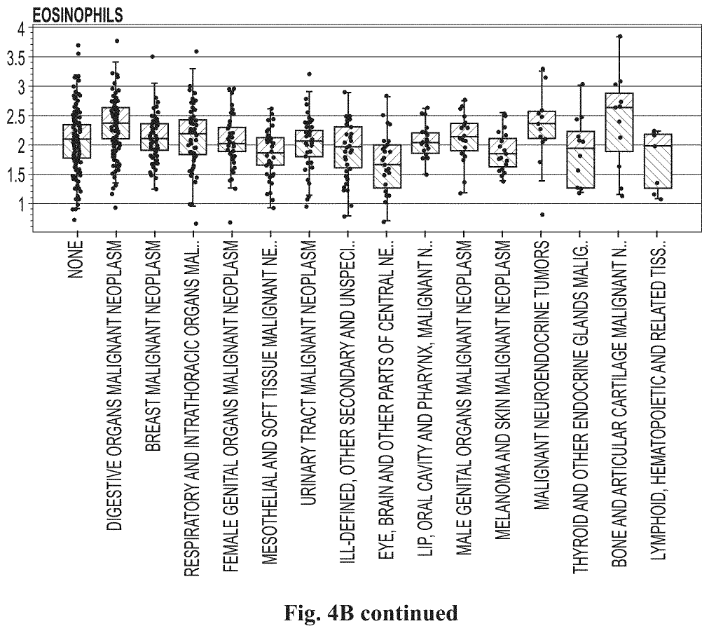

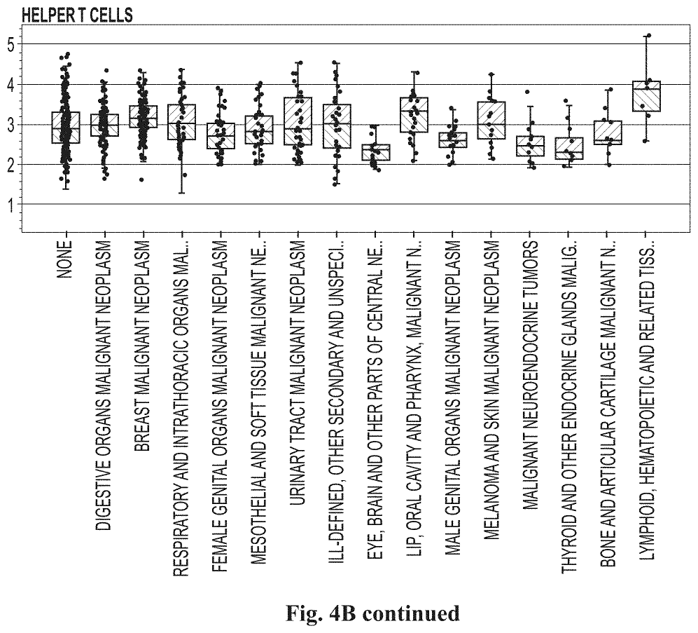

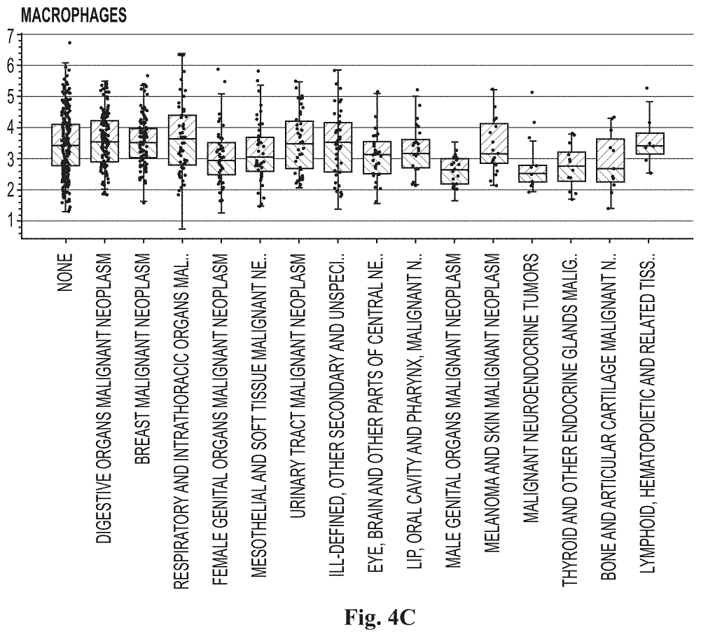

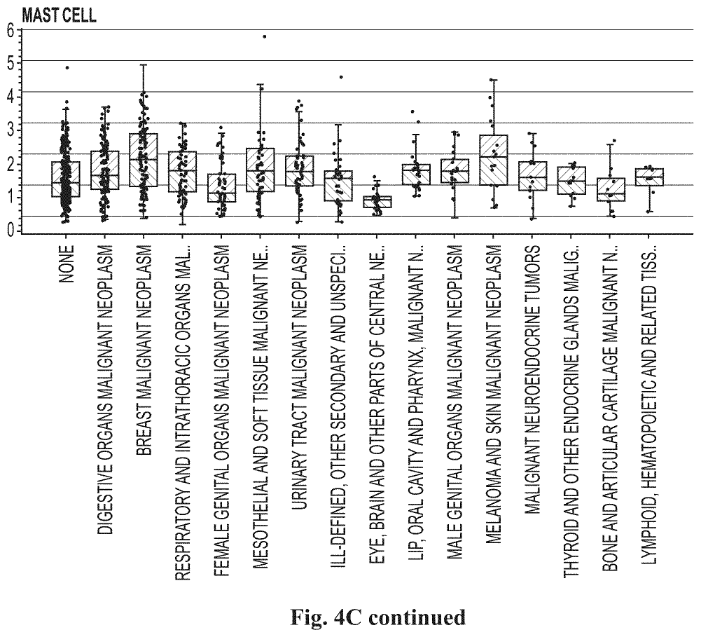

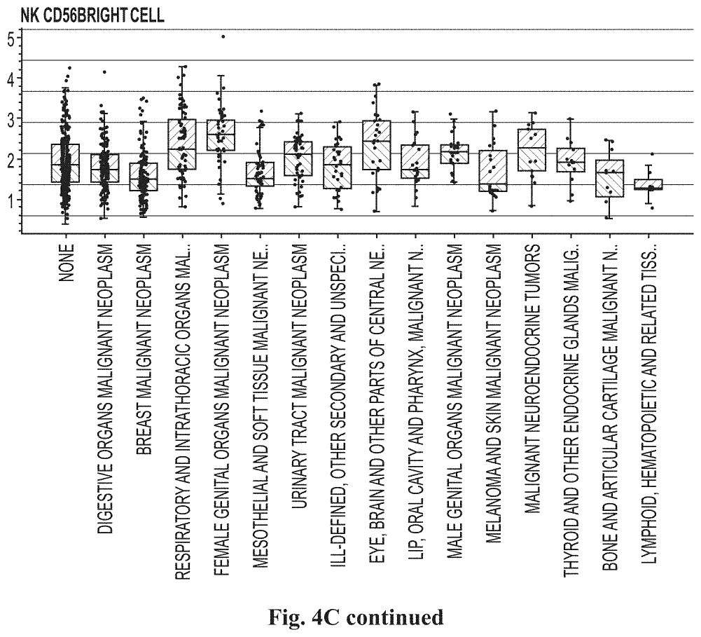

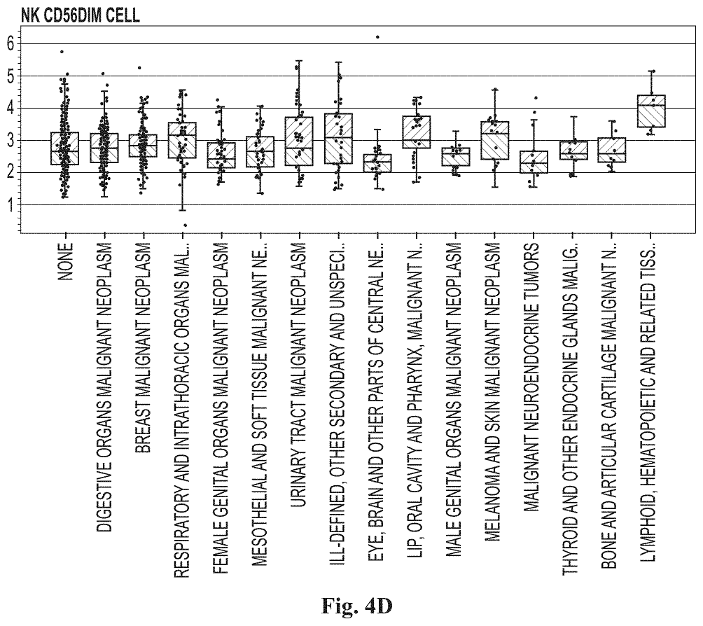

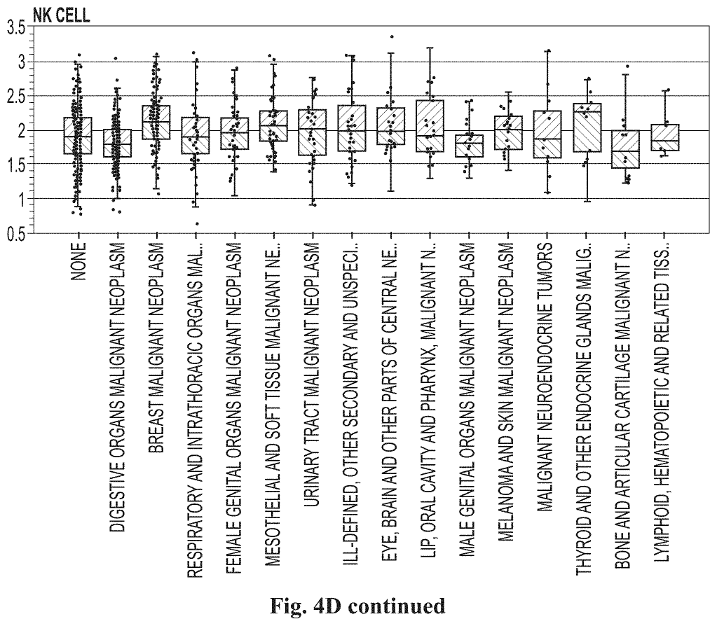

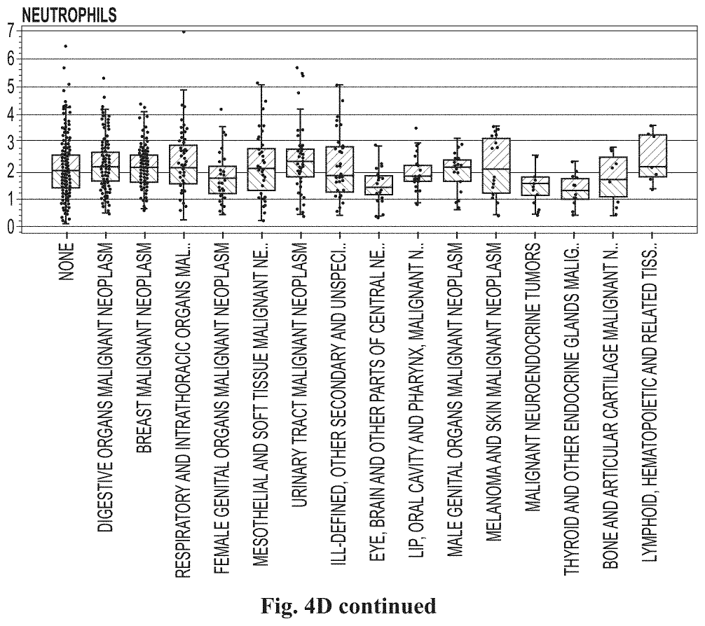

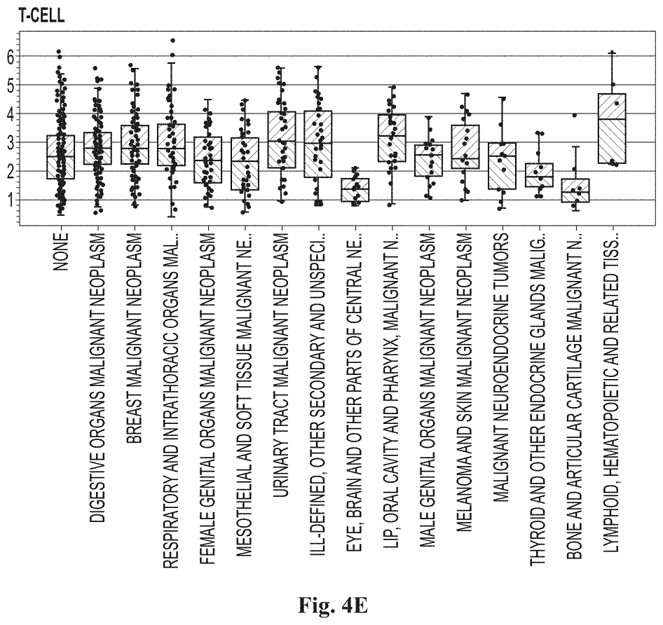

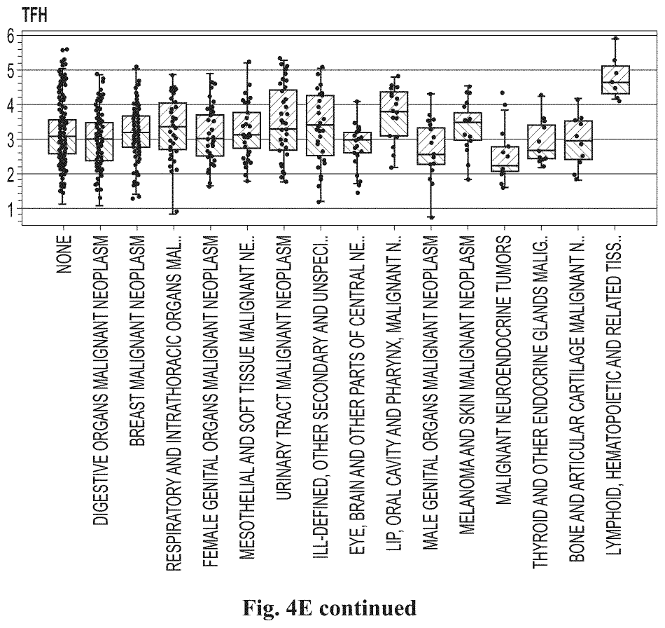

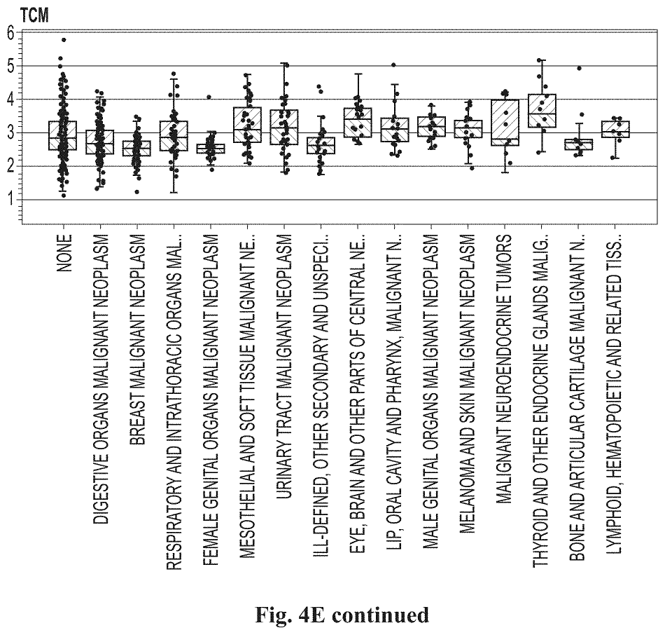

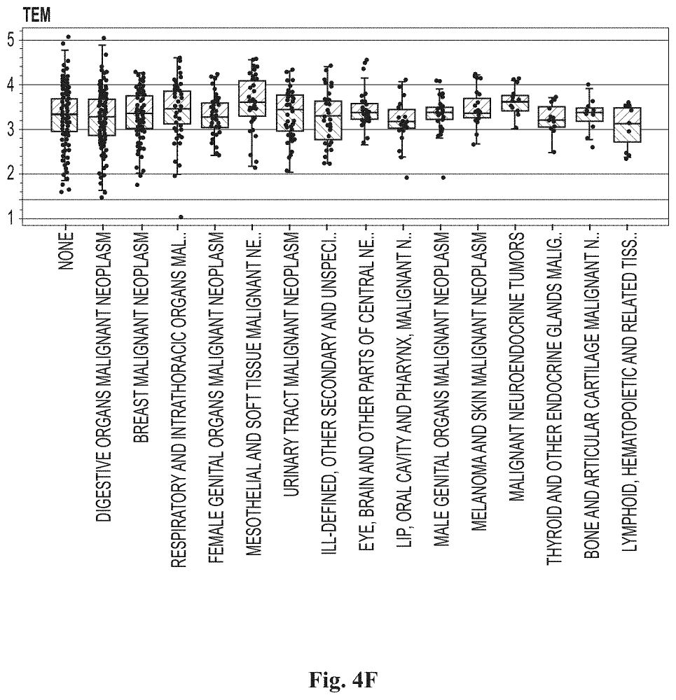

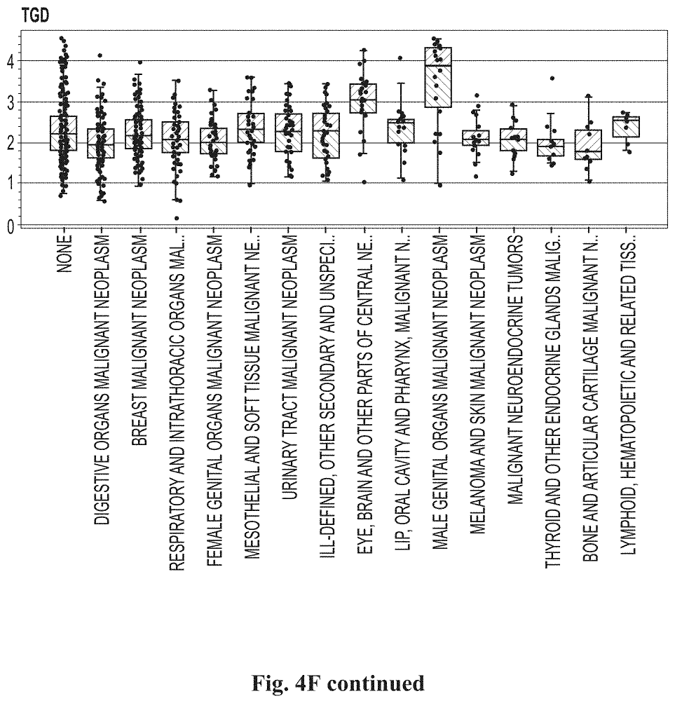

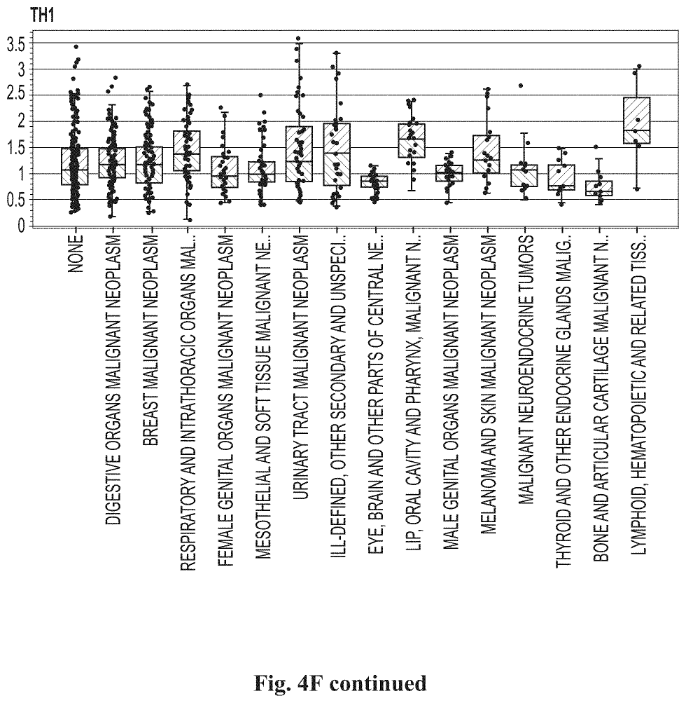

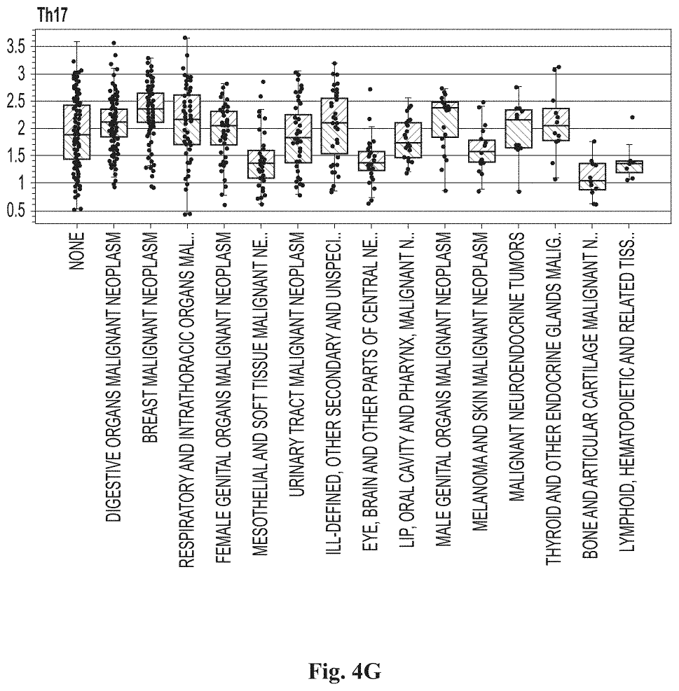

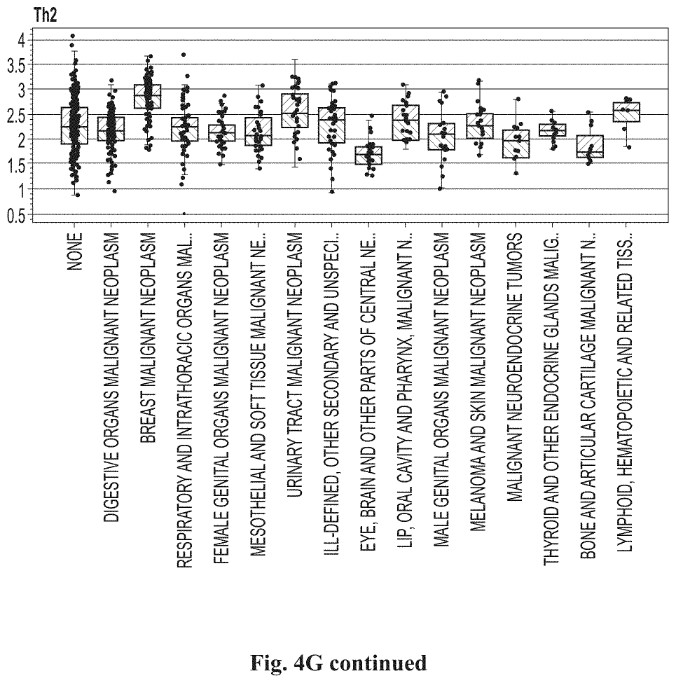

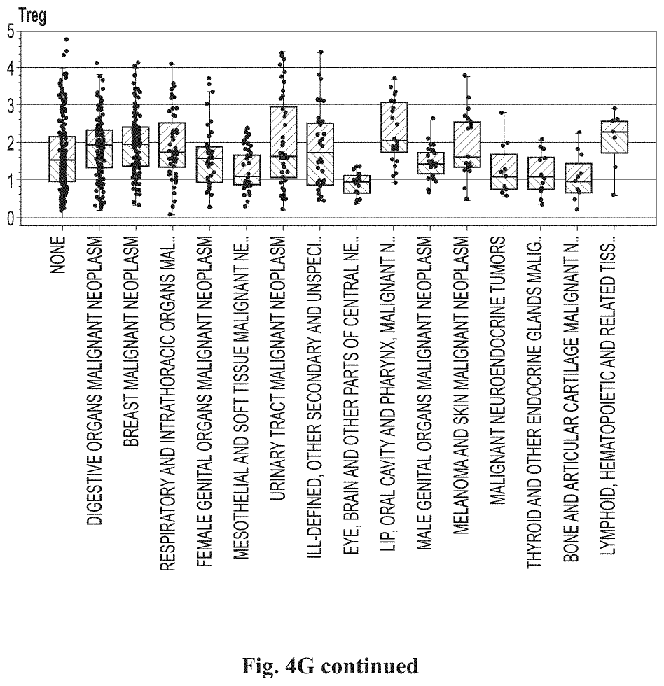

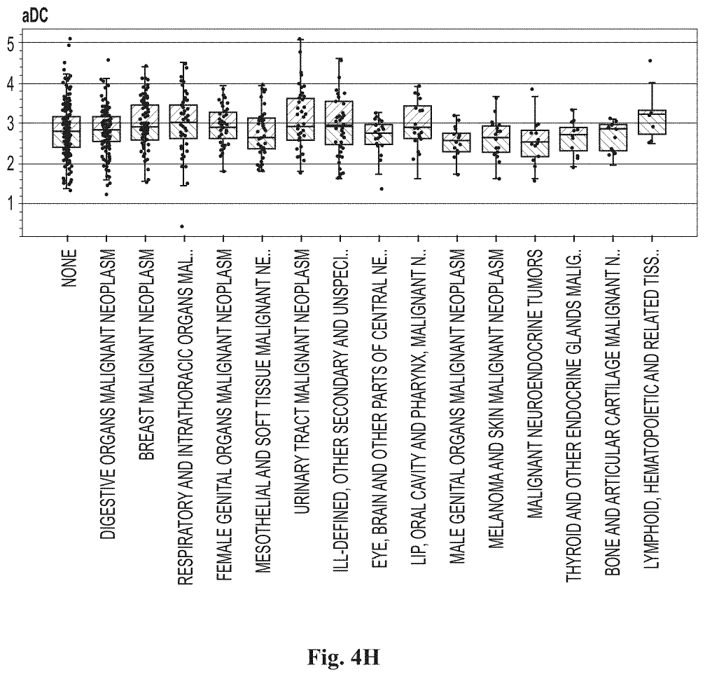

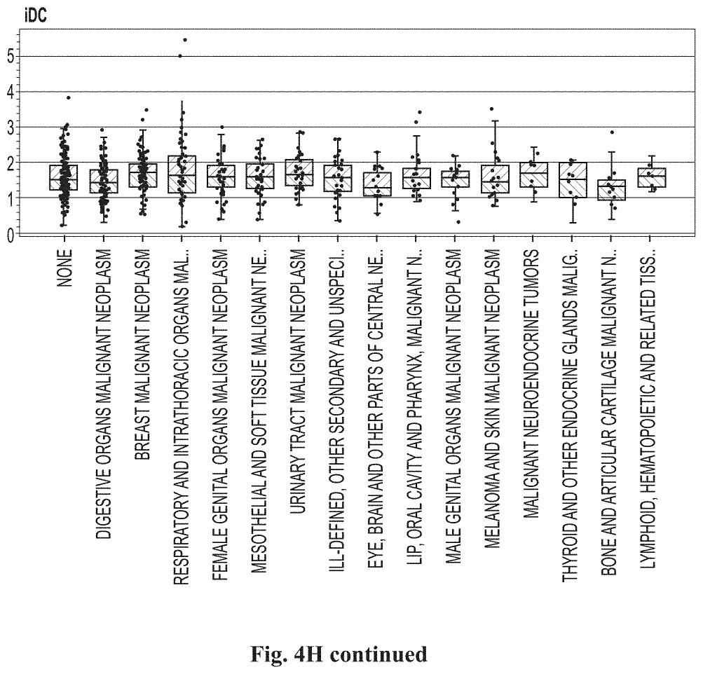

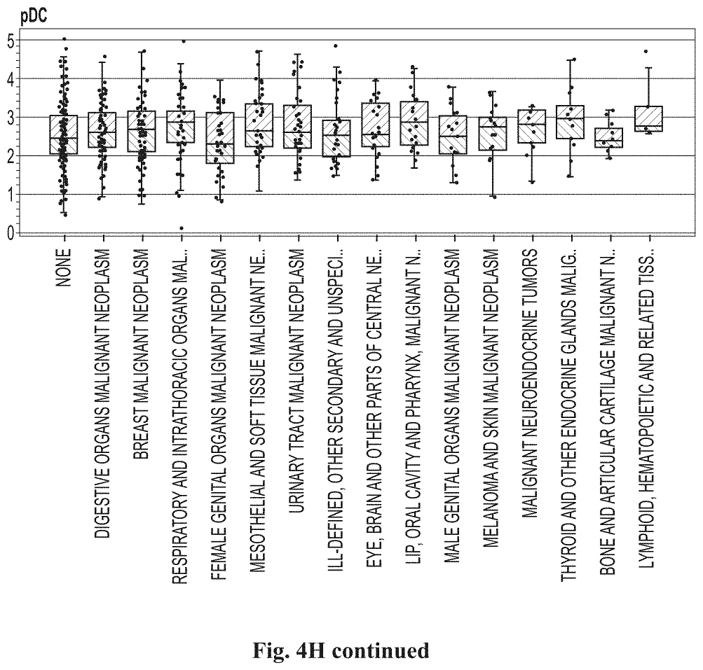

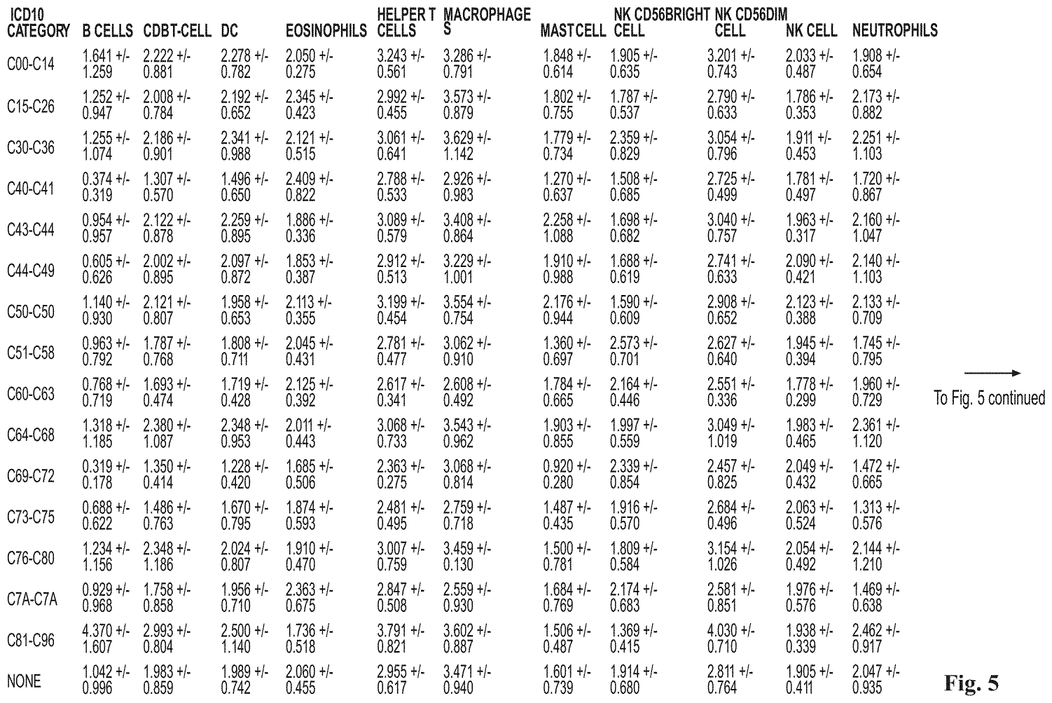

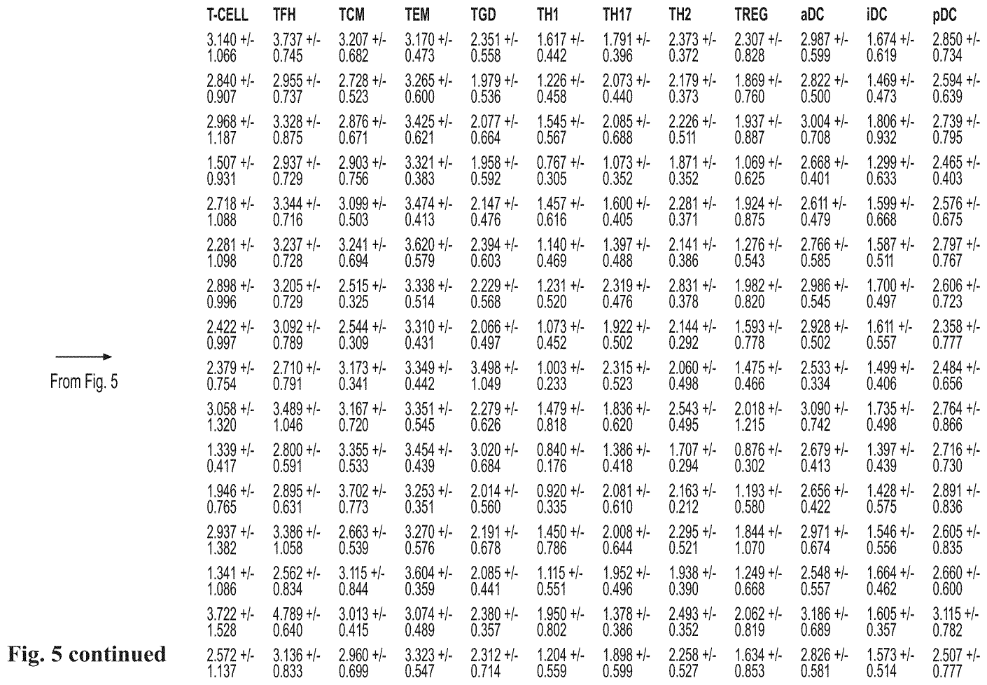

FIGS. 4A-4H provide a more detailed analysis of the immune cell category activation by tissue-type distributions using the same tissue types/ICD10 classification as above, and using log2[tpm+1] expression for all genes in each immune cell category (split up into reported ICD10 categories). The data points are individual reported cases, boxplots are derived from the category (max z=1.5). Cancer type categories are ordered from left-to-right by how many samples were annotated for that cancer type. Notably, as can be seen from the graphs, distinct activation patterns are evident for the particular immune cell type and cancer involved. The inventors then employed statistical analysis for the average gene expression of the particular immune cell and cancer type, and exemplary results are shown in the table of FIG. 5. Here, mean and standard deviation log2[tpm+1] for all genes in each immune cell category are listed, split up into the reported ICD10 category. These statistics are then advantageously used to determine over- (>2 sd), under- (≤2 sd) or normal-activation given a particular tumor tissue type. It should be appreciated that such quantitative analytic process can advantageously be used to correlate gene expression (e.g., as measured by RNAseq) with the presence of specific immune cells in the tumor, and with that to infer whether a tumor is immunologically ‘hot’ or ‘cold’.

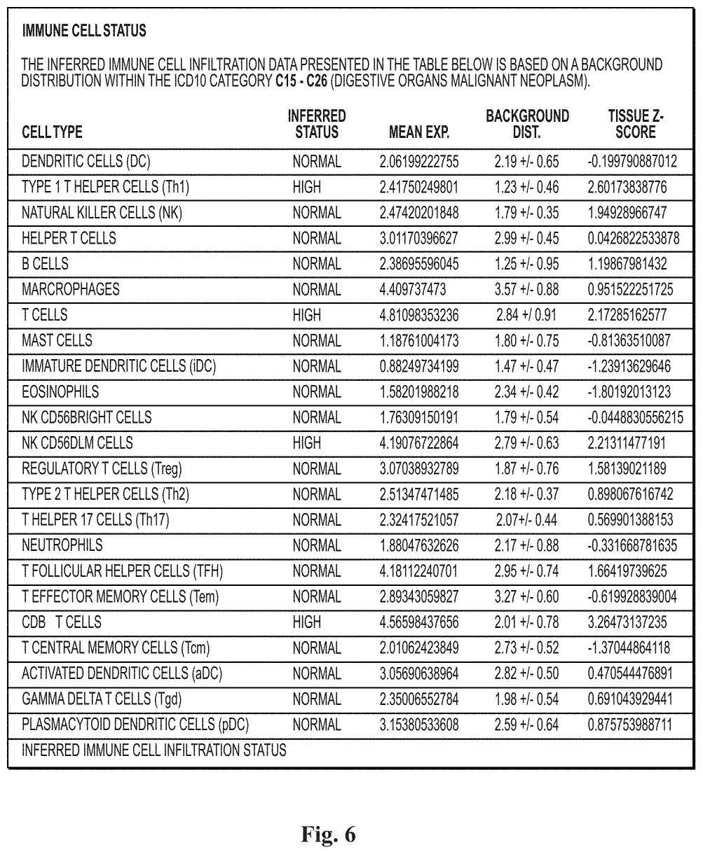

For example, a tumor tissue belonging to ICD10 class C15-C26 (e.g., digestive organs malignant neoplasm) can be analyzed using RNAseq and gene expression data quantified, using the specific tumor tissue type and the tabulated results of FIG. 5. Based on these results, as is exemplarily shown in FIG. 6, immune cell type status/presence can be readily inferred. In the example of FIG. 6, the tumor sample showed activity of Th1 cells, T cells, NK cd56dim cells, and CDB T cells at higher levels. Viewed from a different perspective, it should therefore be recognized that gene expression quantification of specific genes associated with specific immune cells (normalized by tumor tissue type) can be used to infer immune cell presence and/or immune cell activation in the tumor. In this exemplary report format, a shoutout is included that highlights how many immune-cell types are elevated (e.g., 4 elevated signatures).

The inventors further contemplate that transcriptomics data can be obtained from the tumor sample, especially the expression levels of selected genes that are associated with the immune status (e.g., one or more immune marker genes, etc.) to so predict or infer the immune status of the tumor tissue that can implicate potential target for immune therapy. For example, the inventors investigated immune marker co-expression patterns, and particularly checkpoint expression patterns and correlations. FIG. 7 shows exemplary checkpoint expression patterns in three groups: PD-L1 high group (showing high PD-L1 RNA expression levels in the tissue), PD-L1 normal group, PD-L1 low group, respectively. The inventors found that each group shows differential expression patterns of 6 different genes: TIM3, PD-L2, LAG3, PD-L1, IDO1, CTLA4. Here, the expression heatmaps are log2[tpm+1] scale with (blue=0, red>=5), and the colors at the top indicate the different cancer types. Expression heatmaps are ordered by Euclidean distance, and the correlation plots are Pearson correlations (blue=0, red>=0.75). Notably, as can be taken from the unclustered appearance of the cancer type color indicators, there was an apparent lack of significant tissue-dependent expression of immune checkpoint genes. As expected, however, PD-L1 and PD-L2 expression was moderately correlated.

Yet, the inventors found that the transcript expression levels among immune checkpoint genes are often highly correlated. For example, PD-L1 and PD-L2 expression are moderately correlated. In addition, the inventors also found expression levels of IDO and TIM3 are relatively high, particularly in the absence of PD-L1, or when PD-L1 is under-expressed (R=0.78) such that inversely proportional expression correlation to PD-L1 expression. In addition, in low PD-L1 group, LAG3 expression level was also correlated with expression levels of IDO and TIM3. Yet, such proportional correlation could not be clearly seen in high PD-L1 group. Consequently, the data suggest that PD-L1 itself may be a sufficient primary driver of immune suppression (as seen in the PD-L1-high correlation plot) in high PD-L1 group and that IDO1 and TIM3 may play a role in driving immune suppression in low PD-L1 group.

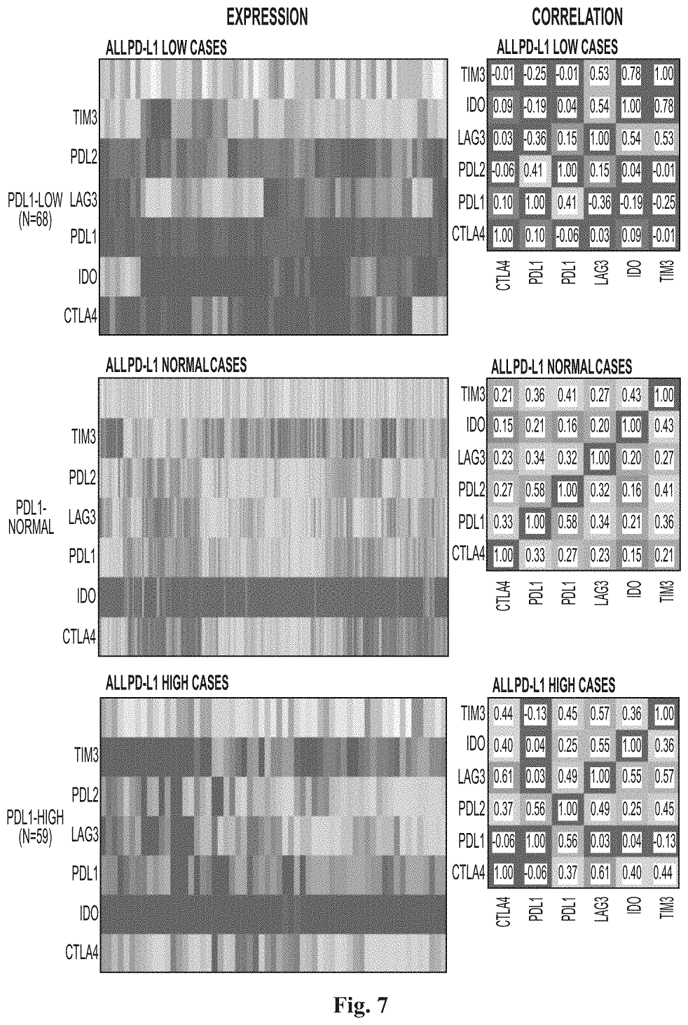

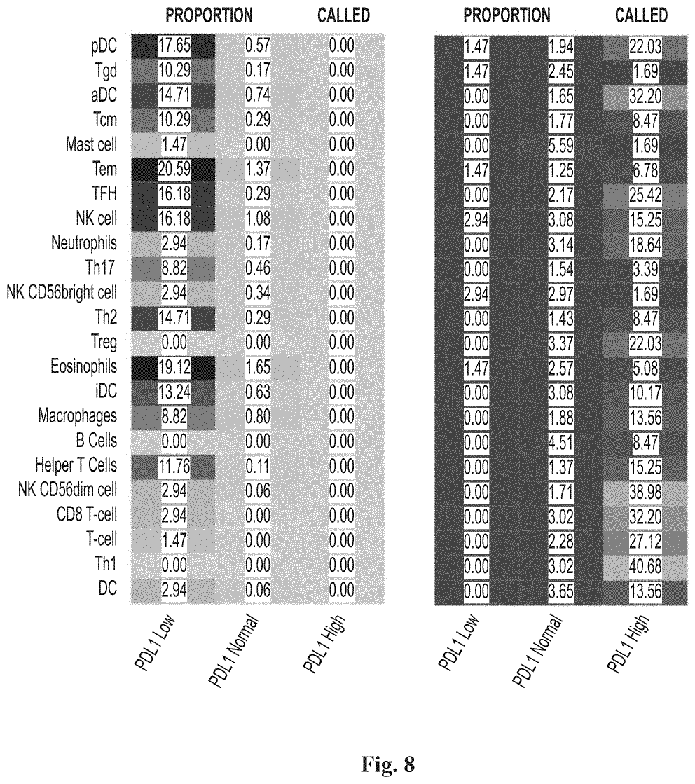

When further investigating the role of PD-L1 with respect to the immune cell categories as noted above, the inventor discovered that the PD-L1 high group is enriched for multiple immune-cell types, including multiple kinds of T-cells and T-helper cells as can be seen in FIG. 8, right plot. Thus, especially in conjunction with the results of the checkpoint expression patterns shown in FIG. 7, it appears that PD-1 expression is probably sufficient to evade these systems. On the other hand, in the PD-L1 low group, CD8 T-Cells, T-Cells, and Th1 cells are significantly under-represented, while most other category of immune cells including NK, and memory T cells showed similar representation as PD-L1 high group. Taken with the results of FIG. 7, the expression data suggest that IDO1 and TIM3 could have a strong role in regulating memory T cells. Thus, in some embodiments, immune cell specific gene expression analysis can be used in predictive analysis of immune therapy, particularly for immune therapy targeting the PD-1/PD-L1 axis, or targeting IDO1 and/or TIM3, based on the PD-L1 expression levels and the immune cell presence patterns.

### Histopathology of Tumor Tissues

The inventors contemplate that while the transcriptomics data of a gene set may provide useful information to infer the immune status or types of immune cells present in the tumor microenvironment, such data may not be sufficient or rather incomplete to infer the accurate immune phenotype of the tumor microenvironment as the locations of the immune cells in the tumor microenvironment are often as critical as the presence of the immune cells with respect to the activity of the immune cells against the tumor cells. For example, even if Th cells are present at or near the tumor microenvironment, Th cells may not be effectively exert their function against the tumor cells, if Th cells are locked in the periphery of the tumor and fail to infiltrate into the core of the tumor tissue.

Thus, in one aspect of the inventive subject matter, the inventors contemplate that distribution of immune cells in the tumor tissue can be determined, and that such distribution data, in combination with the transcriptomics data, can be used to infer the immune phenotype of the tumor. Any suitable sample tissues to obtain distribution data of the immune cell in the tumor are contemplated. Preferably, the sample tissue to obtain distribution data is the substantially same tissue that is used for obtain transcriptomics data. Alternatively, the sample related to the immune status or immune cells is an adjacent tissue that is used for obtain transcriptomics data. In this embodiment, it is preferred that the biopsied tumor tissue are dissected and split into two pieces of tissues facing with each other such that one tissue is used for transcriptomics (and/or for obtaining other types of omics data) and another tissue is used for obtaining distribution data. The tissue surface facing another tissue surface is likely to mostly resemble another tissue's characteristic.

In some embodiments, the distribution of immune cells in the tumor microenvironment can be determined by immunolabeling, either in a live tissue or a fixed and/or frozen tissue, using at least one, preferably at least two, more preferably at least three antibodies that are bound to marker peptides of different types of immune cells (e.g., CD3 (for T cells), CD19 (for B cells), CD56 (for NK cells), CD11C (for dendritic cells), etc.). Any standard or modified immunolabeling technique or protocols can be used including live-cell labeling/imaging. Alternatively and/or additionally, in other embodiments, the distribution of immune cells in the tumor microenvironment can be determined by in situ hybridization (e.g., fluorescence (FISH) or chromogenic (CISH), etc.) using a marker probe binding to cell-type specific expressed RNA or immune-status specific expressed RNA. In addition, tumor cells can be labeled using a tumor cell marker (e.g., common tumor stem cell marker, tumor-specific marker, etc.) or general cell marker (e.g., nucleus marker, eosin staining, etc.) such that the tumor tissue can be distinguished from non-tumor tissue by its structure (e.g., high density core, etc.).

The inventors contemplate that cell distribution data for each cell type detected in the tumor tissue can be analyzed based on the relative location to the core of the tumor mass. Thus, in some embodiments, the distribution data can be analyzed based on the absolute number of immune cells (preferably specific types of immune cells, e.g., T cells, Th cells, etc.) in the core of the tumor mass or in the periphery of the tumor mass (e.g., within 10%, within 20%, within 30%, within 50% of diameter distance from the rim of the tumor mass, etc.) or ratios of immune cells in the core of the tumor mass or in the periphery of the tumor mass. The inventors further contemplate that the number of immune cells and/or ratios of immune cells may be normalized based on the size of the tumor (e.g., volume of the mass, diameter of the tumor mass, etc.) or the tumor cell counts (number of the tumor cells counted in the immuno-labeled tumor tissue or estimated number of tumor cells in the entire tumor mass, etc.) in the tumor mass. In addition, in some embodiments, the number of immune cells and/or ratios of immune cells can be normalized by comparing with a matched normal tissue (non-diseased tissue from the same organ or nearby tissue) or a healthy tissue obtained from a healthy individual (preferably same organ tissue).

Based on the distribution of the immune cells, especially the immune effector cells (e.g., Th cells), the tumor tissue can be classified to immune-absence status, immune-initiation status, immune-active status. Without wishing to be bound by any specific theory, the inventors contemplate that the tumor tissue can be classified to immune-absence status if the number of T cells, especially the number of Th cells, is low in the tumor mass and/or around the tumor mass. Thus, in some embodiments, the classification can be based on the pre-determined threshold on the absolute number of immune cells normalized by the number of tumor cells in the same sample, and/or pre-determined threshold on the ratio of immune cells normalized by the number of tumor cells in the same sample. For example, the tumor tissue can be classified to immune desert status if the number of T cells (preferably Th cells) is less than 20 cells, less than 50 cells, less than 100 cells, less than 200 cells per 0.1 cm², per 1 cm², per 5 cm2 area of the tumor mass and its surrounding no farther than 0.1 mm, 0.5 mm, 1 mm, 2 mm, or 5 mm from a boundary of the tumor mass.

In addition, the inventors contemplate that the tumor tissue can be classified immune-initiation status if the number of T cells, especially the number of Th cells, is normal to high, yet the localization of the T cells are limited to the periphery of the tumor mass. In other words, the inventors contemplate that the tumor tissue can be classified to immune-initiation status if the distribution ratio of the T cells is asymmetrically high in the periphery of the tumor mass compared to the inside of the tumor mass. Thus, for example, the tumor tissue can be classified to immune periphery status if the number of T cells (preferably Th cells) is more than 20 cells, more than 50 cells, more than 100 cells, more than 200 cells per 0.1 cm², per 1 cm², or per 5 cm² area of the tumor mass and its surrounding no farther than 0.1 mm, 0.5 mm, 1 mm, 2 mm, or 5 mm from a boundary of the tumor mass, and the ratio between the T cells inside of the tumor mass and the surrounding of the tumor mass (o farther than 0.1 mm, 0.5 mm, 1 mm, 2 mm, or 5 mm from the tumor mass boundary) is at least 1:2, at least 1:3, at least 1:4, at least 1:5, or at least 1:10.

Further, the inventors contemplate that the tumor tissue can be classified to immune-active status if the number of T cells, especially the number of Th cells, is normal to high and the high percentage of the T cells are localized in the tumor mass. In other words, the inventors contemplate that the tumor tissue can be classified to immune-active status if there are high proportions T cells infiltrated into the tumor mass. Thus, for example, tumor tissue can be classified to immune-active status if the number of T cells (preferably Th cells) is more than 20 cells, more than 50 cells, more than 100 cells, more than 200 cells per 0.1 cm², per 1 cm², or per 5 cm² area of the tumor mass only. In another example, tumor tissue can be classified to immune-active status if the number of T cells (preferably Th cells) is more than 20 cells, more than 50 cells, more than 100 cells, more than 200 cells per 0.1 cm², per 1 cm², or per 5 cm² area of the tumor mass and its surrounding no farther than 0.1 mm, 0.5 mm, 1 mm, 2 mm, or 5 mm from a boundary of the tumor mass, and the ratio between the T cells inside of the tumor mass and the surrounding of the tumor mass (o farther than 0.1 mm, 0.5 mm, 1 mm, 2 mm, or 5 mm from the tumor mass boundary) is at least 1:1, at least 2:1, at least 3:1, at least 5:1, or at least 10:1.

Alternatively and/or additionally, the inventors contemplate that immune status of the tumor tissue can be determined based on the number, ratio, and/or distribution of immune cells other than the effector T cells. For example, immune status of the tumor tissue can be determined immune-resistant if the numbers of Treg cells or other types of immune-inhibitory cells are at least 20 cells, more than 50 cells, more than 100 cells, more than 200 cells per 0.1 cm², per 1 cm², or per 5 cm² area of the tumor mass, and/or ratio between the Treg cells and cytotoxic immune cells (e.g., NK cells, etc.) is lower than 1:10, lower than 1:5, lower than 1:3, lower than 1:1, higher than 2:1, or higher than 3:1. For example, immune status of the tumor tissue can be determined immune-resistant if the number of tumor stem cells in the tumor mass is at least 10 cells, more than 20 cells, more than 50 cells, more than 100 cells per 0.1 cm², per 1 cm², or per 5 cm² area of the tumor mass. Conversely, immune status of the tumor tissue can be determined immune-susceptible if the numbers of Treg cells or other types of immune-inhibitory cells are less than 20 cells, less than 50 cells, less than 100 cells, less than 200 cells per 0.1 cm², per 1 cm², or per 5 cm² area of the tumor mass, and/or ratio between the cytotoxic immune cells (e.g., NK cells, etc.) and Treg cells is higher than 2:1, higher than 3:1, or higher than 5:1.

Consequently, the inventors contemplate that an immune phenotype of a tumor tissue can be more comprehensively and thoroughly identified using both transcriptomics data and the cell distribution data. In some embodiments, the immune phenotype of a tumor tissue can be inferred by identifying the cell types and/or and activity of immune cells or immune-inhibitory cells per location of the tumor tissue (e.g., inside tumor mass, surrounding the tumor mass, distant from the tumor mass, etc.) and mapping the cell types in the tumor tissue. For example, increased or relatively high expression levels of genes associated with Th cell and the increased number of Th cells in the tumor tissue visualized by immunolabeling can be corroboratively and/or collectively used to confirm the increased Th cell recruitment to the tumor tissue. In addition, further increased or substantially high expression levels of genes associated with Th cells in view of the increased number of Th cells in the tumor tissue may indicate increased activation of Th cell in or around the tumor mass. In another example, increased or relatively high expression levels of genes associated with one type of immune-inhibitory cells not associated with a specific, single marker can be corroborated with histopathology data of a plurality of similar immune-inhibitory cells to infer the location, activity level, and/or percentage of the specific types of immune-inhibitory cell in the tumor tissue.

Consequently, it should be appreciated that omics (and especially transcriptomics) data can be used to not only determine the immune cell type in a tumor, but also to determine the activity of an immune cell in a tumor. For example, while conventional histopathology analysis could readily detect the presence of CD8 T cells in a tumor tissue, such analysis will not readily allow determination of the T cell as being activated, suppressed, or anergic. Similarly, NK cells may be readily identified by antibody staining, but cytotoxic activity of such cells is typically not easily determined using staining processes. Still further, omics (and especially transcriptomics) data can also be used to identify pathway activities in immune cells and/or tumor cells, which adds further detailed information of the tumor and/or immune cell status.

The inventors also contemplate that inferring the immune phenotype of a tumor tissue using the combination of transcriptomics data and cell distribution data can be corroborated and/or supplemented by analysis of genomics data set and/or proteomics data set of the tumor tissue that may include the protein expression levels, post-translational modification of the proteins, and inferred protein activity. It is contemplated that overexpressed transcript of a gene may not necessarily correlate to an increased activity of a protein encoded by a gene or an increased effect of such proteins to the cell or to the tissue. For example, the overexpressed transcript of a mutated protein may produce a dominant negative effect of a signaling pathway in which the mutated protein is placed. In another example, even if the overexpressed transcript of a gene leads to overexpression of a protein, the protein may not be active due to loss of post-translational modification necessary to generate active form of protein (e.g., phosphorylation, glycosylation, etc.). In still another example, even if the overexpressed transcript of a gene leads to production of active proteins, those proteins may be mislocalized in the cell (e.g., intracellular localization instead of cell membrane, failed to be secreted, etc.) such that the activity of the protein may not lead to the effect of the protein to the cell or tissue.

Thus, in some embodiments, the inventors contemplate that one or more, preferably two or more types of omics data sets (e.g., genomics data, transcriptomics data, proteomics data, etc.) can be used to infer a pathway characteristic of the tumor cells and/or immune cells in the tumor tissue to construct a further thorough immune phenotype or signature of the tumor tissue. While any suitable methods of analyzing pathway characteristics of cells are contemplated (e.g., GSEA, SPIA, PathOlogist, ARACNE, MINDy, CONEXIC, NetBox, Mutual Exclusivity Modules in Cancer (MEMo), etc.), a preferred method uses PARADIGM (Pathway Recognition Algorithm using Data Integration on Genomic Models), which is a genomic analysis tool described in WO2011/139345 and WO/2013/062505 and uses a probabilistic graphical model to integrate multiple genomic data types on curated pathway databases. For example, a pathway model having a plurality of pathway elements (e.g. DNA sequence, RNA sequence, protein, protein function) can be accessed and a protein function or activity in the pathway can be inferred in a function of regulatory parameters in pathways using PARADIGM. In this example, each pathway elements are aligned along the path, preferably in DNA-RNA-protein-protein activity format, having a regulatory node that controls activity along the path as a function of a plurality of regulatory parameters. The regulatory parameter may vary depending on the regulatory node connecting the pathway elements. For example, where the pathway element comprises a DNA sequence and the regulatory parameter is a transcription factor, a transcription activator, a RNA polymerase subunit, a cis-regulatory element, a trans-regulatory element, an acetylated histone, a methylated histone, and/or a repressor. Where the pathway element comprises a RNA sequence and the regulatory parameter is an initiation factor, a translation factor, a RNA binding protein, a ribosomal protein, an siRNA, and/or a polyA binding protein. Where the pathway element comprises a protein and the regulatory parameter is a phosphorylation, an acylation, a proteolytic cleavage, and an association with at least another protein.

In some embodiments, a plurality of pathway models, each to infer an activity of a protein encoded by the DNA of the pathway, can be coupled to form a signaling pathway model of a tumor cell, an immune cell or a tumor tissue. In some embodiments, the signaling pathway may include an immune-stimulatory pathway (e.g., NK cell activation pathway, T cell activation pathway, etc.) immune-inhibitory pathway (e.g., Treg activation pathway, etc.) and/or immune-resistant pathway (e.g., tumor stem cell development pathway, immune evasion pathway, etc.). Additionally, such signaling pathway can be also characterized as a constitutively activated pathway, a functionally impaired pathway (e.g., due to reduced expression or reduced activity of a protein in the signaling pathway, etc.), and/or a dysregulated pathway (e.g., due to overexpressed, mutated protein that are dominant-negatively impact the signaling pathway, etc.), based on the inferred protein activity of one or more coupled pathway models.

The inventors contemplate that such obtained pathway characteristic of the tumor cells, immune cells and/or tumor tissue can be used to assign a molecular signature of the tumor. In some embodiments, tumor A, B, and C can be assigned to different molecular signatures where each tumor shows at least one or more different pathway characteristics in relation to the tumor cell activity and/or immune cell activity. In other embodiments, portions of a tumor can be assigned to different molecular signatures based on the different pathway characteristics determined locally. In such embodiments, it is contemplated that the omics data can be obtained locally in the tumor tissue (e.g., center of the tumor mass, periphery of the tumor mass, etc.) using a local dissection method (e.g., laser microdissection, tissue microdissection punching, etc.).

It should be appreciated that the contemplated methods of using a plurality of data sets analyzing the immune status of the tumor tissue from different perspectives or angles to allow more thorough characterization of the tumor tissue by taking account of a large number of factors that could not be included if the analysis uses only one type of omics data (e.g., transcriptomics data only, etc.). In addition, combination of the omics data analysis with histology data of the tumor tissue allows to correlate the cell type and activity level of the cell type with the location such that the effect of the cell to the tumor tissue can be more accurately inferred (i.e., active NK cells or effector T cells inside the tumor mass would be more effective than the those active cells present in the periphery of the tumor mass to attach the tumor cells, etc.).

Based on the obtained omics data set(s) and/or histology data of the tumor tissue, the immune phenotype of the tumor tissue can be determined. In some embodiments, the immune phenotype of the tumor tissue can be classified into at least three, at least four, or at least 5 different stages depending on the numbers, activities, and/or distribution of the immune cell relative to the tumor cells, and also the numbers and activities of the tumor cells in the tumor tissue. For example, the immune phenotype of the tumor tissue can be classified into three stages: immune desert phenotype, immune-periphery phenotype, and inflammatory phenotype. The immune desert phenotype can be characterized with a lack of pre-existing immunity, evidenced by one or more of low expression levels of effector immune cells (e.g., T cell) associated genes, low expression level of immune cell activation signaling pathway associated genes (in either transcriptomics data or proteomics data), and low number of immune cells distributed in the tumor mass as well as the tumor periphery. The immune-periphery phenotype can be characterized with the initiation of, yet not active, immune response in the tumor tissue, evidenced by increased (higher than normal compared to matched normal tissue) levels of effector immune cells (e.g., T cell) associated genes, increased expression level of immune cell activation signaling pathway associated genes (in either transcriptomics data or proteomics data), and increased number of immune cells distributed in the tumor periphery, yet scarce distribution of the immune cells, especially the effector immune cells, in the tumor mass. Thus, in this phenotype, it is expected that immune cells in the tumor tissue, while active, is not yet effective to induce immune response against the tumor. The inflammatory phenotype can be characterized with the active immune response against the tumor cells in the tumor tissue, evidenced by increased (higher than normal compared to matched normal tissue) levels of effector immune cells (e.g., T cell) associated genes, increased expression level of immune cell activation signaling pathway associated genes (in either transcriptomics data or proteomics data), and increased number of immune cells distributed in the tumor mass, implicating that the immune cells are recruited and penetrated (or infiltrated) into the tumor mass.

As multiple factors and data set may be used to determine the immune phenotype(s) of the tumor tissue, the inventors contemplate that the immune phenotype(s) of the tumor tissue can be determined using a matrix of a plurality of factors identified by various types of data sets and/or a scoring method. For example, in some embodiments, in which transcriptomics data set and the histology data set are used to determine the immune phenotype of a tumor tissue, a portion of the transcriptomics data in relation to the immune cell identity and the activity level can be placed in a matrix (e.g., expression level of 10 different genes, 20 different genes, 30 different genes, etc.) in one axis (e.g., x-axis), and the histology data in relation to the immune cell identity and distribution of such immune cells can be placed in the matrix in one or more axis (e.g., numbers of different immune cells (e.g., 1-10 Th cells/per predetermined area, 10-20 Th cells/per predetermined area, 20-30 Th cells/per predetermined area, 1-10 NK cells/per predetermined area, 10-20 NK cells/per predetermined area, 20-30 NK cells/per predetermined area, etc.) in y-axis, distance of the distributed cell from the center of the tumor mass in z-axis, etc.). In such embodiments, the immune phenotype(s) of the tumor tissue can be determined based on the pattern of the data distributed in the matrix.

Additionally and/or alternatively, each factor in the matrix can be assigned with a score, and the immune phenotype(s) of the tumor tissue can be determined based on the value calculated with those scores. For example, each expression level of different gene can be scored between 1-3 depending on the relative expression level (e.g., high, normal, low, etc.), and inferred activity of the immune cells in the tumor tissue can be calculated based on each score of the expression level of different genes and additional score based on the synergistic effects or antagonistic effects of expression levels of genes. Further, the distribution of the immune cell can be scored based on the type, distance from the tumor mass, and the numbers of such immune cells. For example, the effector T cell distribution score can be calculated based on the type (e.g., each effector T cell in the tumor tissue is assigned to score 5, etc.), distance from the tumor mass (50% less score if the effector T cell is located outside of the tumor mass, 70% less score if the effector T cell is located farther than 10% of the radius distance from the outer rim of the tumor mass, etc.), and the numbers of the effector T cells (e.g., ×20 score if there are 20 effector T cells located in the similar area, etc.). All those scores can be taken together to calculate the final score and the immune phenotype(s) can be determined based on the final score (e.g., immune desert phenotype if the final score is less than 30, immune periphery phenotype if the final score is between 30-100, etc.).

Using the immune phenotype(s) determined from the plurality of data sets on the tumor tissue, a treatment regimen to the patient can be generated and/or updated, and/or the patient can be treated with such generated or updated treatment regimen. For example, where the patient's tumor tissue is determined to be immune desert phenotype, the treatment regimen may include any drugs or treatments to prime T cell activation, recruitment, and/or priming the exposure of T cells to the antigens via enhancing antibody presentation. Thus, exemplary treatment regimen for the immune desert phenotype tumor includes a cancer vaccine (e.g., virus, bacteria, yeast vaccine) that carries tumor antigens (e.g., HER2, EGFR, ALK, MEK, HDAC, etc.) or neoepitopes (e.g., patient- and tumor-specific neoantigen) so that T cells can be primed by contacting the antigen presenting cells presenting such tumor antigens or neoepitopes. Another exemplary treatment regimen for the immune desert phenotype tumor may include agents that activate tumor cells including CEA and/or CD3. In another example, where the patient's tumor tissue is determined to be immune periphery phenotype, the treatment regimen may include any drugs or treatments that can stimulate the immune cells to infiltrate into the tumor mass, including, but not limited to, cytokines and/or chemokines for recruiting T cells to the tumor mass (e.g., VEGF, CXCR4, etc.). In still another example, where the patient's tumor tissue is determined to be inflammatory, the treatment regimen may include any drugs or treatments that can enhancing the T cell activity, including, but not limited to any checkpoint inhibitors (e.g., PD-L1/PD-1 inhibitor, a TIM3 inhibitor or an IDO1 inhibitor, etc.), cytokines or chemokines (e.g., VEGF, CSF, etc.), or T cell activating molecule (e.g., CEA, CD3, etc.).

From the different perspective, the inventors also contemplate that an outcome of a tumor treatment can be predicted based on the tumor's immune phenotype. For example, the likelihood of success of immune therapy including one or more checkpoint inhibitors may be predicted high if the immune phenotype of the tumor tissue is determined to be inflammatory, and conversely may be predicted low if the immune phenotype of the tumor tissue is determined to be immune desert. In another example, the likelihood of success of immune therapy including at least one of a vaccine composition and an immune stimulatory cytokine is predicted high if the immune phenotype of the tumor tissue is determined to be immune periphery or immune desert.

In one exemplary method, the inventors combined digital masking using deep-neural nets with transcriptomic deconvolution to infer where immune-subpopulations may reside in the TME. More specifically, an unselected set of 187 clinical samples from the ImmunityBio database were analyzed. Each sample had H&E stained diagnostic slides with pathologist-annotated tumor regions, as well as deep whole-transcriptomic sequencing (>200M reads). Deep neural networks previously trained on TCGA slide images were used to generate digital spatial masks for 3 characteristics: tumor-content, lymphocytes, and stroma. Patients were scored based on the presence of intratumoral lymphocytes (iTIL) and stromal lymphocytes (sTILs). Immune subpopulations were then inferred from RNAseq expression of published immune-cell-specific genesets (Bindea, 2013 & Danaher, 2011), as was Wnt-signaling level (Slattery, 2018). Significant associations between immune subpopulations and level of infiltration were analyzed.

Using such approach, manually annotated positive tumor regions were accurately digitally masked as >83% tumor or lymphocyte. Wnt signaling was strongly associated with overall stromal content (Rho=0.47, p<0.0001). Strong anti-correlation was observed between levels of sTILs and iTILs (Rho=−0.42, p<0.0001), and remained significant when including overall stroma area as a covariate. Digital lymphocyte masks somewhat correlated with RNAseq-based deconvolution of lymphocyte classes (Rho=0.30, p=0.0001) in line with reports from others (Rosenthal, 2019), however, this decreased when comparing lymphocyte count within annotated tumor regions only (Rho=0.17, p=0.03), despite high concordance of lymphocyte counts within and outside of annotated regions overall (Rho=0.82, p<0.0001). RNAseq-based lymphocyte levels were more associated with sTILs than iTILs (Rho=0.19 vs. −0.28, p<0.01 respectively). It was found that adaptive response effectors such as NK and T-cells were found more resident in surrounding stromal tissue than infiltrating tumor tissue. Moreover, increased Wnt/B-catenin signaling in stromal regions, reported by others as immunosuppressive, may sequester immune effectors and aid in immune escape.

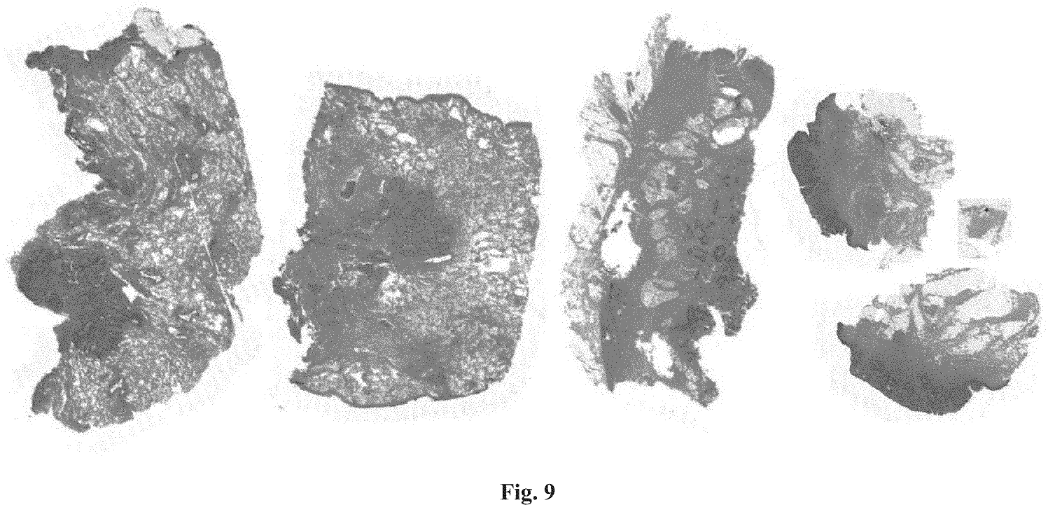

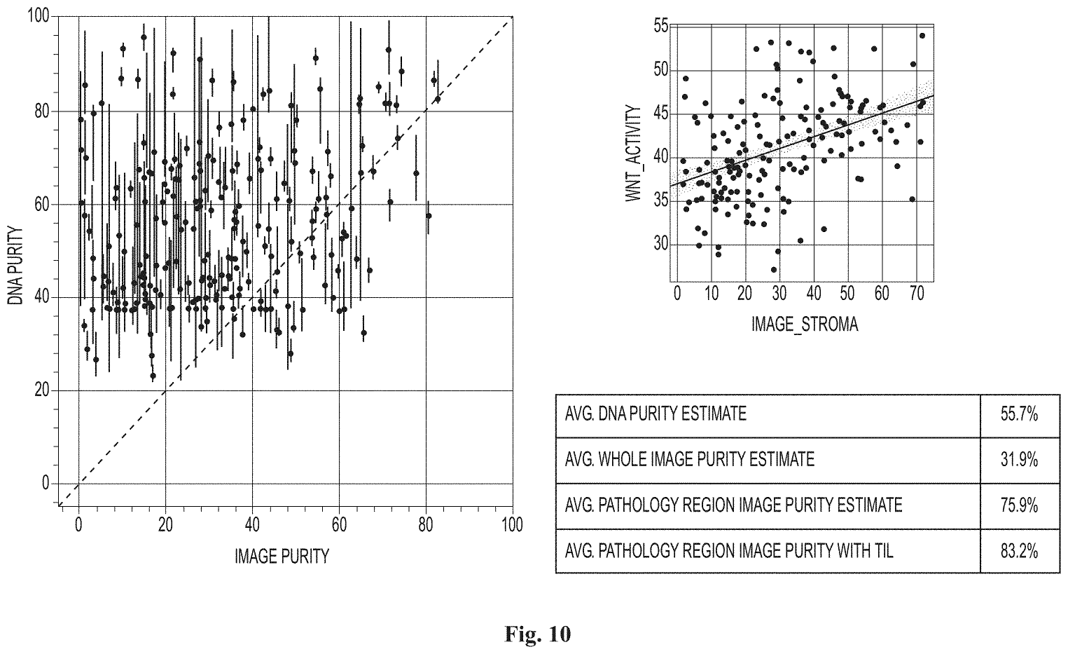

FIG. 9 depicts example images with high specificity between deep-net generated tumor masks and pathologist annotations. Tumor predictions are marked in orange, lymphocytes in green, stroma in purple, pathologist annotations in blue/red circles. FIG. 10 shows that purity and stromal estimates align with expectations from DNA & RNA. More specifically, as can be seen in the left graph a comparison of 243 NGS-based purity estimates and deep-net-based purity estimates is illustrated where the X-axis denotes percentage of tiles classified as tumor, and the y-axis denotes purity estimates from GPSCancer. Lines indicate min-max range of estimates from DNA sequencing. Notably, overall DNA purity estimates are higher than image-based estimates, most likely because of macrodissection prior to sequencing as well as different cell-density between tumor and non-tumor regions. The table to the right provides summary statistics for DNA and deep-net based purity. On average DNA purity is ˜24% higher. Despite overall having much lower average purity estimation from deep nets, regions marked as at least 80% tumor by pathologists in 184 images were also masked as 83% tumor regions on average by deep-nets (when allowing lymphocytes to be marked as correct). The smaller graph demonstrates that stromal tissue is reported to express Wnt-pathway genes. As can be taken from the graph, there is a highly significant correlation between Wnt geneset activation (inferred based on 32 Wnt-associated genes in URL: www.ncbi.nlm.nih.gov/pmc/articles/PMC5814196/) and deep-net predicted stromal area.

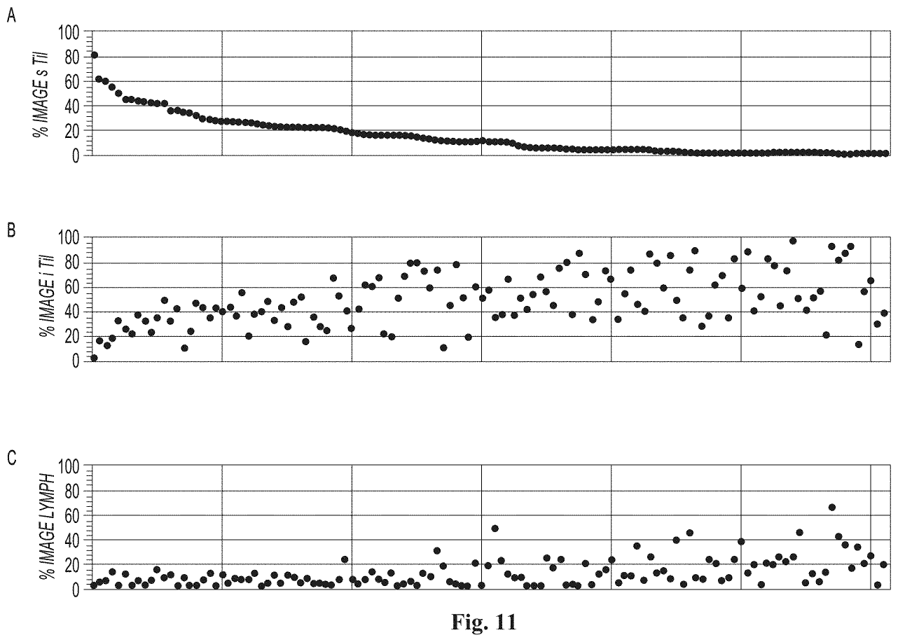

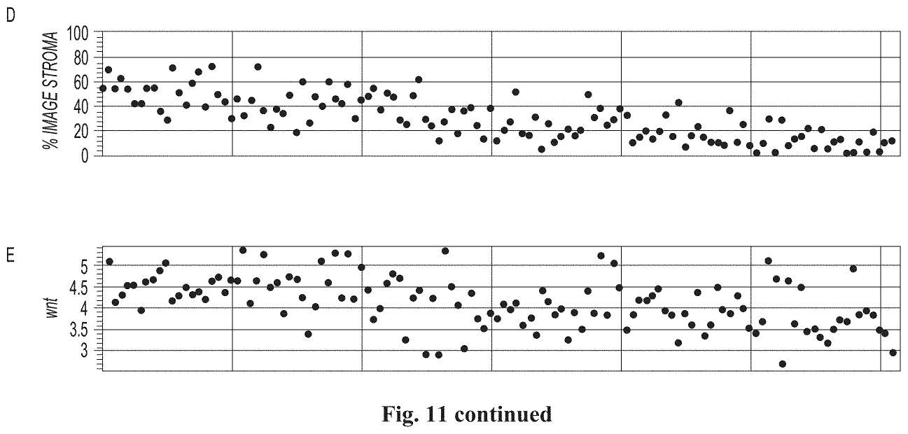

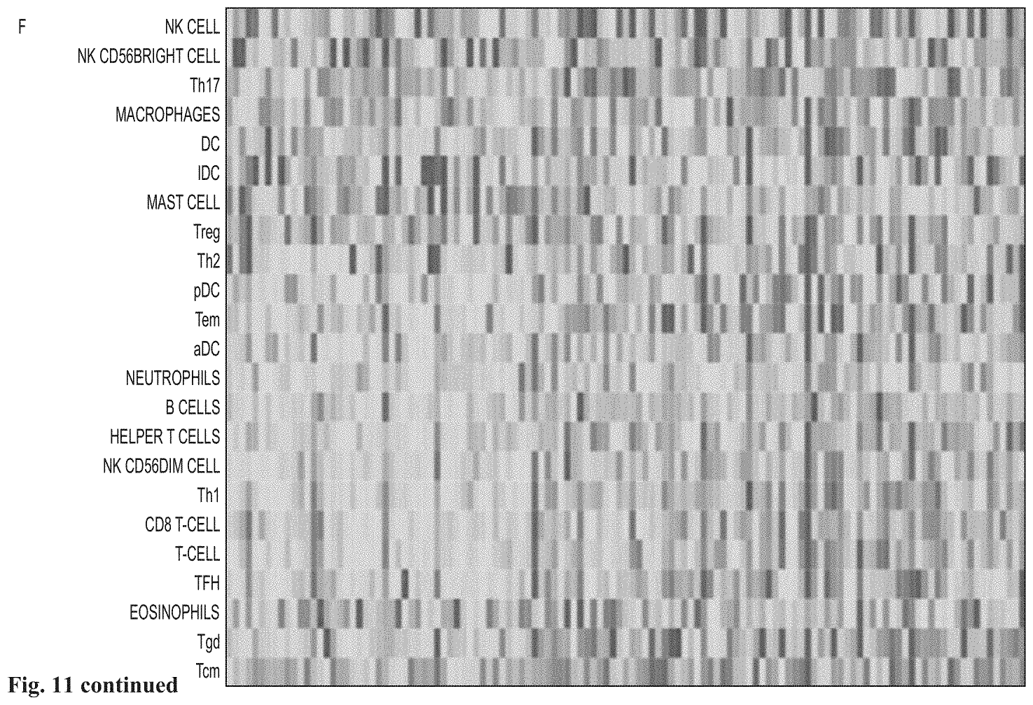

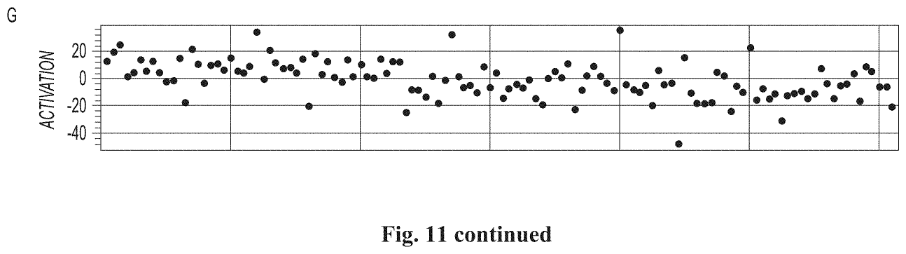

The inventors further performed RNAseq-based immune deconvolution sorted by sTIL levels. Here, analyzed samples were filtered from 184 to the 166 samples that had >15% tumor area as an image quality filter, and FIG. 11 depicts exemplary results. Panel A) depicts the percentage of lymphocyte regions also classified as within stromal regions (sorted). Panel B) depicts the percentage of lymphocyte regions also classified as within tumor regions. Note these are somewhat anti-correlated with sTILs. Panel C) shows the percentage of all slide patches that classify as lymphocyte-rich. Note that variance in % lymphocyte increases as sTIL decreases. Panel D) shows the percentage of all slide patches that classify as stroma. Note this is somewhat correlated with sTIL levels, and Panel E) depicts Wnt geneset activation. Note that this (like % stroma) is also somewhat correlated with sTIL. Panel F) shows a Heatmap of inferred activities (z-scores) for 23 immune-cell types, based on comparison to a background population. In general the ‘hotter’ samples are associated with higher sTILs. Finally, Panel G) depicts the sum of Z-score across all immune-cell types (i.e. sum of columns in F). Note that activation level somewhat correlates with sTILs.

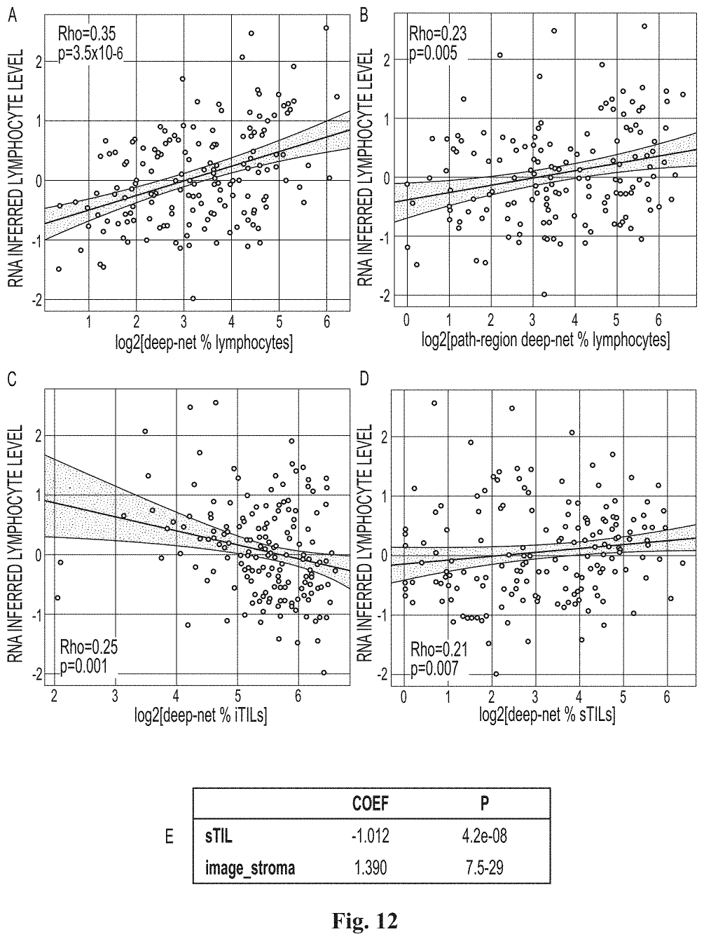

FIG. 12 illustrates exemplary results for RNAseq-based lymphocyte score vs. various deep-net assessments. RNA-based estimate is the mean z-score for NK,T, and B cells. More specifically, Panel A shows the correlation between RNA estimates of total lymphocyte and image estimates is ˜0.35, in line with what others have presented (e.g. Rosenthal et al, 2019). Panel B) depicts the correlation between RNA and lymphocyte count goes down when only assessing pathologist-annotated tumor regions, suggesting the positive correlation in A) is mainly driven by areas outside tumor regions (as annotated by pathologists). Panel C) shows RNA-based estimates and iTIL is significantly anticorrelated, suggesting RNA levels aren't driven by lymphocytes in tumor-regions. Panel D) illustrates that RNA estimates are somewhat correlated with sTILs. This statistically supports that RNA-based estimates of immune infiltration are potentially driven by stromal or non-tumor-infiltrating lymphocytes. Table E) shows coefficients and p-values for a bivariate linear regression model relating iTIL ˜sTIL+image_stroma. Notably, even when taking overall stroma level into consideration, sTIL percentage is still a strong contraindicator of tumor infiltration.

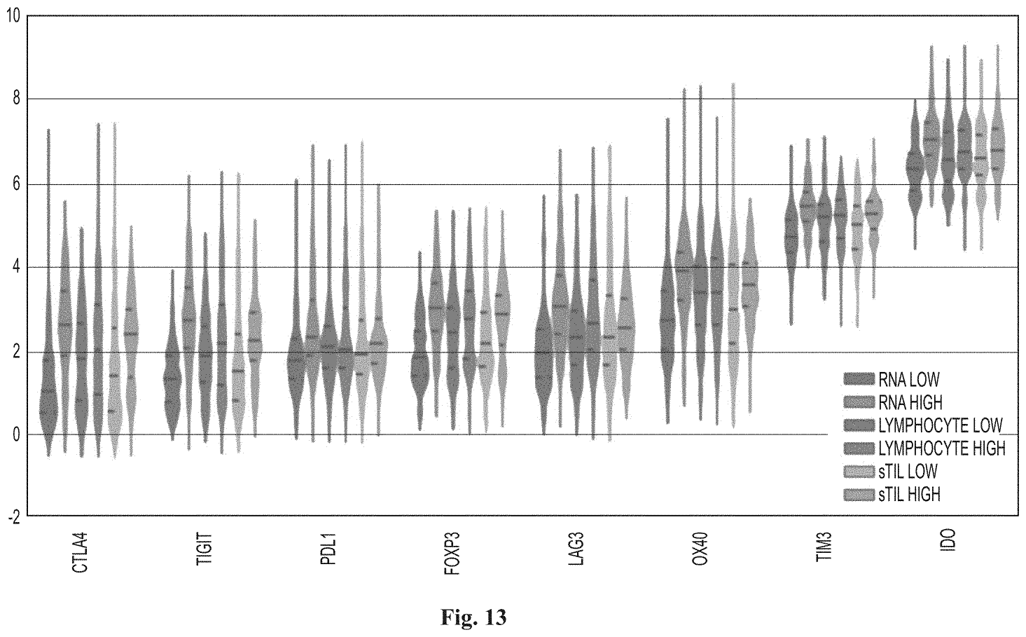

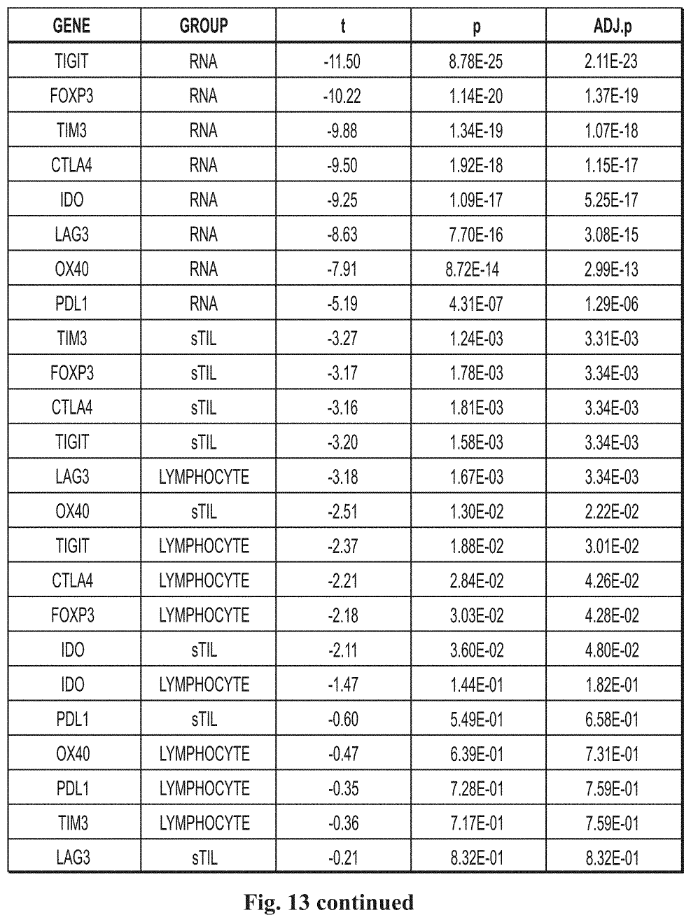

FIG. 13 demonstrates exemplary results for checkpoint expression patterns using different methods of bifurcating patients. RNA expression of 8 key immunoregulatory (TO) molecules, split on either RNAseq-based lymphocyte score median (blue/green), image % lymphocyte patches (red/purple), or sTIL (i.e. percentage of lymphocytes within stroma) (yellow, cyan). t-test results for each gene between these 3 methods to group patients are presented in the table on the right, with p-values adjusted using Benjamini-Hochberg multiple hypothesis correction. Higher immune infiltration by RNA-based deconvolution is significantly associated with elevated levels of all IO genes. sTIL level is significantly associated with differential expression of most IO genes. Lymphocyte area is the least informative of the three grouping methods shown here, although it is significantly associated with 4/8 IO genes analyzed.

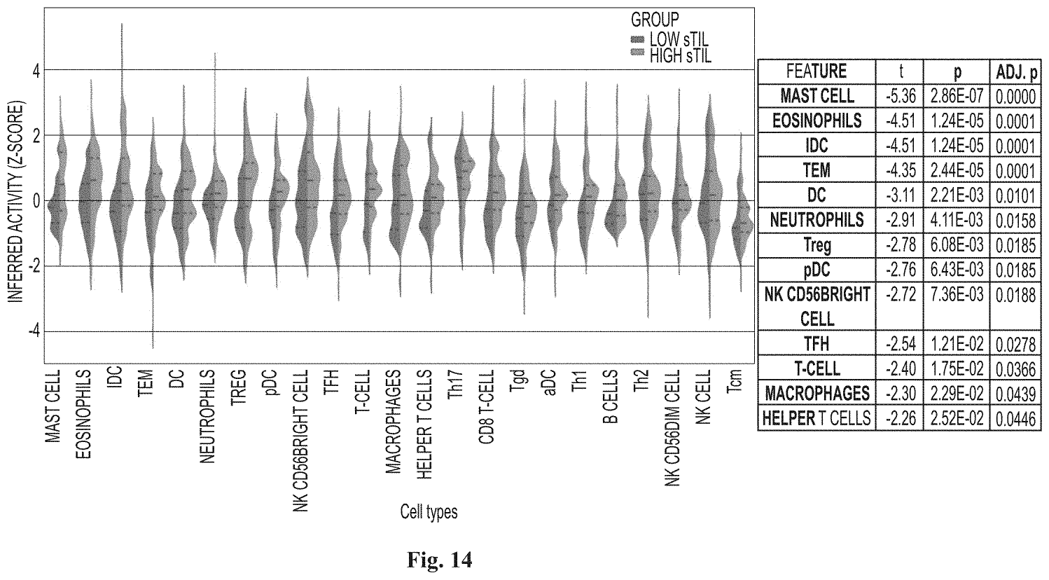

FIG. 14 exemplarily illustrates which immune-cell types are most associated with sTIL levels (and expression of most checkpoints). Shown on the left are violin plots contrasting inferred activity levels of each immune cell set with high sTIL (top 50%) vs. low sTIL (bottom 50%). Shown on the right is a table of the associations between sTIL and immune-cell type that remain significant after Benjamini-Hochberg adjustment. All cell types were higher in high sTIL vs. low sTIL (split by median sTIL score). Mast cells are most significantly associated with high sTILs, and are known to reside in connective tissue which stroma resembles. Very diverse immune cell types seem to be associated with sTIL levels, suggesting this is a general measure of immune-competency rather than a specific response-type, although perhaps independent of PDL1-mediated evasion.

Based on the above, it should therefore be recognized that deep-net tumor/normal mask performs as expected against pathologist's gold-standard, and that RNA deconvolution and that deep-net lymphocyte scores agree as much as expected, in line with results from others. Moreover, it was shown that Wnt signaling (RNA) correlates with deep-net stromal content, that RNAseq-based ‘immune-hot’ scores correlate with stromal content, specifically sTILs, and not iTILs, and that patients with high sTILs appear to have a wide variety of immune-cell modalities elevated. Finally, it can be concluded that increased Wnt/B-catenin signaling in stromal regions, reported by others as immunosuppressive, may sequester immune effectors and aid in immune evasion.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1-34. (canceled)

35. A method of treating a patient having a tumor, comprising:
quantifying or obtaining expression levels for a plurality of distinct genes in the tumor, wherein the distinct genes are associated with respective distinct types of immune cells;
determining distribution of at least one type of the immune cells in the tumor;
inferring an immune phenotype of the tumor based on the expression levels for the plurality of distinct genes and the distribution of the at least one type of the immune cells; and
treating the patient with an immune therapy selected based on the immune phenotype.

36. The method of claim 35, wherein the distinct immune cells in the tumor are selected from pDC, aDC, TfH, NK cells, neutrophils, Treg, iDC, macrophages, T helper cells, CD8 T cells, CD4 T cells.

37. The method of claim 35, wherein the expression level is measured via qPCR or RNAseq.

38. The method of claim 35, wherein the plurality of distinct genes and the respective distinct types of immune cells is listed in the table of FIG. 1.

39. The method of claim 35, further comprising determining over-expression or under-expression for each of the distinct genes relative to respective reference ranges, wherein the reference ranges are specific for a specific tumor type, or wherein the over-expression or under-expression is determined when the quantified expression level exceeds +/−2SD of the reference range.

40. (canceled)

41. The method of claim 35, wherein the reference ranges are specific for a specific tumor type as classified in ICD10.

42. The method of claim 35, wherein the distribution of at least one type of the immune cells is determined from immunolabeling or in situ hybridization of the tumor.

43. The method of claim 35, further comprising:
obtaining at least two of genomics, transcriptomics, and proteomics data from tumor cells in the tumor; and
inferring a pathway characteristic of the tumor cells;
assigning a molecular signature of the tumor cells based on the pathway characteristic.

44. The method of claim 43, wherein the pathway characteristic comprises a constitutively activated pathway, a functionally impaired pathway, and a dysregulated pathway.

45. The method of claim 43, wherein the pathway characteristic comprises an immune-inhibitory pathway and immune-resistant pathway.

46. The method of claim 43, wherein the pathway characteristic is inferred using PARADIGM.

47. The method of claim 43, wherein the immune phenotype is further inferred based on the pathway characteristic.

48. The method of claim 35, wherein the immune phenotype comprises immune desert, immune periphery, and immune infiltration.

49. The method of claim 35, wherein the immune therapy includes a checkpoint inhibitor when the immune phenotype is determined to be immune desert.

50. The method of claim 49, wherein the checkpoint inhibitor is a PD-L1/PD-1 inhibitor, a TIM3 inhibitor or an IDO1 inhibitor.

51. The method of claim 35, wherein the immune therapy includes a chemokine to recruit T cells when the immune phenotype is determined to be immune periphery.

52. The method of claim 51, wherein the chemokine is VEGF or CXCR4.

53. A method of using transcriptomics data of a plurality of distinct genes and immune cell distribution data of a tumor of a patient to characterize a tumor, to predict treatment outcome for immune therapy of the tumor, or to treat the patient, wherein the plurality of distinct genes are associated with respective distinct types of immune cells, and wherein the method comprises inferring an immune phenotype of the tumor based on the transcriptomics data and immune cell distribution data.

54. The method of claim 53, wherein the transcriptomics data comprise expression levels for the plurality of distinct genes obtained using RNA-seq.

55. (canceled)

56. The method of claim 53, wherein the immune cell distribution data is obtained from immunolabeling or in situ hybridization of the tumor.
